(19) United States
(12) Patent Application Publication
(10) Pub. No.: US 2008/0305994 A1
(43) Pub. Date: Dec. 11, 2008

(54) Pharmaceutical Compositions and Method for Treating, Reducing, Ameliorating, Alleviating, or Preventing Dry Eye

(76) Inventors: Jinzhong Zhang, Pittsford, NY (US); Zhenze Hu, Pittsford, NY (US); Keith Wayne Ward, Ontario, NY (US)

Correspondence Address: Bausch & Lomb Incorporated, One Bausch & Lomb Place, Rochester, NY, 14604-2701, US

(21) Appl. No.: 12/124,599

(22) Filed: May 21, 2008

Related U.S. Application Data
(60) Provisional application No. 60/942,842, filed on Jun. 8, 2007.

(51) Int. Cl. A61K 38/13; A61K 31/155; A61K 31/4709; A61K 31/517; A61K 31/4365; A61P 27/02

(52) U.S. Cl. 514/11; 514/633; 514/313; 514/312; 514/266.21; 514/291

(57) ABSTRACT

A composition for treating, reducing, ameliorating, alleviating, or preventing a dry eye condition or an opthalmologic disorder that has an etiology in inflammation comprises an inhibitor of activity of poly(ADP-ribose) polymerase (“PARP”). The composition can also include a modulator of pro-inflammatory gene expression.

### CROSS REFERENCE

This application claims the benefit of Provisional Patent Application No. 60/942,842 filed Jun. 8, 2007 which is incorporated by reference herein.

### BACKGROUND

The present invention relates to pharmaceutical compositions for the treatment, reduction, amelioration, alleviation, or prevention of a dry eye condition or a disorder that requires the rewetting of the eye. In particular, the present invention relates to pharmaceutical compositions that comprise an inhibitor of a poly(ADP-ribose) polymerase (“PARP”) for the treatment, reduction, amelioration, alleviation, or prevention of dry eye syndrome. In addition, the present invention relates to a method for treating, reducing, ameliorating, alleviating, or preventing the dry eye syndrome using such an inhibitor of PARP.

Dry eye, also known as keratoconjunctivitis sicca (“KCS”), is a common opthalmologic disorder affecting millions of people each year. In general, dry eye conditions result from decreased tear production, excessive tear evaporation, or abnormality in mucin or lipid components of the tear film. Dry eye conditions can be caused by a variety of factors. There has been increasing evidence that inflammation may be an important factor in the pathogenesis of KCS. For example, inflammation of the lacrimal and meibomian glands can curb production of the aqueous and lipid components of the tear film, respectively. In addition, elevated levels of pro-inflammatory mediators, including IL-1, have been detected in the conjunctival tissues of patients afflicted with systemic autoimmune diseases, such as Sjögren's syndrome. See; e.g., U.S. Patent Application Publication 2006/0058277; or http://www.mayoclinic.com/health/dry-eyes/DS00463 (visited Apr. 16, 2007). These patients also suffer with severe dry eye. Sjögren's syndrome is a chronic disorder in which white blood cells, recruited by the pro-inflammatory mediators, attack the moisture-producing glands, such as lacrimal and salivary glands, resulting in their degeneration and inducing their apoptosis. Active T-cell infiltrate in the conjunctiva also has been reported in non-Sjögren's syndrome dry eye. See; e.g., M. E. Stern et al., Invest. Ophthalm. & Vis. Sci., Vol. 43, No. 8, 2609 (2002). Dry eye may afflict individuals with differing severity. In mild cases, a patient may experience burning, a feeling of dryness, and other symptoms of ocular discomfort. In severe cases, vision may be substantially impaired. Although dry eye may have a variety of unrelated pathogenic causes, they all share as a common effect the breakdown of the ocular tear film, with dehydration of and subsequent damage to the exposed outer ocular surfaces, which can lead to apoptosis of ocular epithelial cells. See; e.g., S. Yeh et al., Invest. Opthalmol. & Vis. Sci., Vol. 44, No. 1, 124 (2003).

Production of pro-inflammatory mediators is under the control of several important nuclear transcription factors that are activated by yet other nuclear enzymes. Poly(ADP-ribose) polymerase-1 (“PARP-1,” also known as poly(ADP-ribose) synthetase or poly(ADP-ribose) transferase), one member of a family of six nuclear enzymes discovered to date, has been shown to regulate the expression of various proteins at the transcriptional level. Of special importance is the regulation by PARP-1 of the production of pro-inflammatory mediators such as the inducible nitric oxide synthase (“iNOS”), intercellular adhesion molecule I (“ICAM-1”), and major histocompatibility complex class II (“MHC-II”). NF-κB is a key nuclear transcription factor in the regulation of these pro-inflammatory mediators, and PARP-1 has been shown to act as a coactivator in the NF-κB-mediated transcription. Although there is currently no consensus in the literature regarding whether the modulation of NF-κB-mediated transcription assisted by PARP-1 universally requires the catalytic activity of PARP-1, there is ample evidence resulting from studies of PARP-1 inhibitors that such catalytic function is necessary for the pathogenesis of inflammation in several types of tissues. See; e.g., L. Virag and C. Szabo, Pharmacological Review, Vol. 54, No. 3, 375-429 (2002). For example, secondary damage to initially uninjured neurons, resulting from excitotoxic brain injury accounts for most of the infracted area and the loss of brain function after stroke. One major component of secondary neuronal damage is the migration of macrophages and microglial cells toward the site of injury, where they produce large quantities of toxic cytokines and oxygen radicals, which result in further damage to the surrounding tissues. PARP-1 is highly activated in phagocytosing microglial cells, but not in resting microglia. This inflammatory process is strongly controlled by expression of the integrin CD11a, regulated by PARP-1 through the formation of a nuclear PARP/NF-κB protein complex. It was demonstrated that down regulation of PARP-1 or CD11a abrogated microglial migration almost completely and protected neurons from secondary damage. See S. D. Skaper, Ann. N.Y. Acad. Sci., Vol. 993, 217 (2003). In another investigation into the role of PARP-1 in inflammation, data supported a conclusion that PARP-1 activation plays a significant role in the development of acute respiratory distress syndrome, a severe form of lung inflammation. Pharmacological inhibition of PARP-1 resulted in significant downregulation of TNF-α and MIP-1α, and tended to reduce IL-6 production. Pharmacological inhibition of PARP-1 also resulted in significant reduction in the myeloperoxidase activity in the lung, an indication of reduction in the accumulation of neutrophils. L. Liaudet et al., Am. J. Respir. Crit. Care Med., Vol. 165, 372 (2002). In addition, it was demonstrated that inhibition of PARP by 3-aminobenzamide reduced the lipopolysaccharide (“LPS”)-induced leukocyte recruitment within pulmonary arterioles, capillaries, and venules, and suppressed expression of intercellular adhesion molecule 1 (“ICAM-1”) in rabbits. Thus, inhibition of PARP can help to control inflammation. R. Kiefinann et al., Am. J. Physiol. Lung Cell. Mol. Physiol., Vol. 285, L-996 (2003).

Prior-art therapies for dry eye have included both palliative agents, such as artificial tear formulations, and drugs, such as topical steroids, topical retinoids (e.g., Vitamin A), oral pilocarpine, and topical cyclosporine. In general, the palliative therapies are capable of providing short-term relief from some of the symptoms of dry eye, but frequent application of the palliative products to the eye is required to maintain this relief, since these products generally do not eliminate the physiological sources of the dry eye conditions. The drug therapies that have been proposed in the prior art have had limited success in treating dry eye conditions. One reason for the limited efficacy of prior-art drug therapies has often been attributable to the inability of the drug to eliminate or reduce the root causes of the dry eye conditions. Steroidal drugs also can have side effects that threaten the overall health of the patient.

It is known that certain glucocorticoids (also referred to herein as “corticosteroids”) have a greater potential for elevating intraocular pressure (“IOP”) than other compounds in this class. For example, it is known that prednisolone, which is a very potent ocular anti-inflammatory agent, has a greater tendency to elevate IOP than fluorometholone, which has moderate ocular anti-inflammatory activity. It is also known that the risk of IOP elevations associated with the topical ophthalmic use of glucocorticoids increases over time. In other words, the chronic (i.e., long-term) use of these agents increases the risk of significant IOP elevations. Unlike bacterial infections or acute ocular inflammation associated with physical trauma, which requires short-term therapy on the order of a few weeks, dry eye conditions require treatment for extended periods of time, generally several months or more. This chronic use of corticosteroids significantly increases the risk of IOP elevations. In addition, use of corticosteroids is also known to increase the risk of cataract formation in a dose- and duration-dependent manner. Once cataracts develop, they may progress despite discontinuation of corticosteroid therapy.

Chronic administration of glucocorticoids also can lead to drug-induced osteoporosis by suppressing intestinal calcium absorption and inhibiting bone formation. Other adverse side effects of chronic administration of glucocorticoids include hypertension, hyperglycemia, hyperlipidemia (increased levels of triglycerides) and hypercholesterolemia (increased levels of cholesterol) because of the effects of these drugs on the body metabolic processes.

Therefore, there is a continued need to provide improved pharmaceutical compositions to treat, reduce, ameliorate, alleviate, or prevent the dry eye condition. It is also very desirable to provide novel compositions that avoid at least an adverse side effect of prior-art glucocorticoid-based compositions used to treat, reduce, ameliorate, alleviate, or prevent the same condition.

### SUMMARY

In general, the present invention provides pharmaceutical compositions for treating, reducing, ameliorating, alleviating, or preventing in a subject a dry eye condition or other disorders that require rewetting of the eye (for example, disorders that require restoring normal tear function).

In one aspect, a pharmaceutical composition of the present invention comprises an inhibitor of PARP activity (hereinafter sometimes referred to as “PARP inhibitor”), in an amount effective for treating, reducing, ameliorating, alleviating, or preventing a dry eye condition or disorder in a subject.

In another aspect, such PARP comprises PARP-1.

In still another aspect, the pharmaceutical composition comprises an inhibitor of PARP activity, wherein the PARP inhibitor is attached or binds to, forms a complex or associates with another molecule (“carrier molecule”).

In yet another aspect, said another molecule (carrier molecule) can enhance a delivery of the PARP inhibitor to a target tissue in the subject.

In still another aspect, a pharmaceutical composition of the present invention comprises an ophthalmic topical formulation, injectable formulation, or implantable formulation or device.

In a further aspect, a method for treating, reducing, ameliorating, alleviating, or preventing a dry eye condition or other disorders that require rewetting of an eye, comprises administering into a subject a composition that comprises an inhibitor of PARP activity, in an amount and at a frequency sufficient to treat, reduce, ameliorate, alleviate, or prevent said dry eye condition or said disorders.

In still another aspect, a method for treating, reducing, ameliorating, alleviating, or preventing a dry eye condition or other disorders that require rewetting of an eye, comprises administering into a subject a composition that comprises: (a) an inhibitor of PARP activity; and (b) a modulator of a pro-inflammatory gene expression; in an amount and at a frequency sufficient to treat, reduce, ameliorate, alleviate, or prevent said dry eye condition or said disorders.

Other features and advantages of the present invention will become apparent from the following detailed description and claims.

### DETAILED DESCRIPTION

As used herein, an inhibitor of PARP activity includes, but is not limited to, a compound, agent, or material that at least reduces an activity of PARP in a pharmacologically or physiologically meaningful magnitude, which activity comprises a catalytic or enzymatic activity, or an ability of PARP to associate or bind to another molecule, or a combination thereof.

In one aspect, said another molecule can be a peptide, protein, or polynucleic acid.

In another aspect, a reduction in an activity of PARP can be demonstrated in an in vitro assay.

In general, the present invention provides pharmaceutical compositions for treating, reducing, ameliorating, alleviating, or preventing in a subject a dry eye condition or other disorders that require rewetting of the eye (for example, disorders that require restoring normal tear function). In one aspect, such a condition or disorder has an etiology in chronic inflammation.

Glucocorticoids (“GCs”) are among the most potent drugs used for the treatment of allergic and chronic inflammatory diseases. However, as mentioned above, long-term treatment with GCs is often associated with numerous adverse side effects, such as diabetes, osteoporosis, hypertension, glaucoma, or cataract. These side effects, like other physiological manifestations, are results of aberrant expression of genes responsible for such diseases. Research in the last decade has provided important insights into the molecular basis of GC-mediated actions on the expression of GC-responsive genes. GCs exert most of their genomic effects by binding to the cytoplasmic GC receptor (“GR”). The binding of GC to GR induces the translocation of the GC-GR complex to the cell nucleus where it modulates gene transcription either by a positive (transactivation) or negative (transrepression) mode of regulation. There has been growing evidence that both beneficial and undesirable effects of GC treatment are the results of undifferentiated levels of expression of these two mechanisms; in other words, they proceed at similar levels of effectiveness. Although it has not yet been possible to ascertain the most critical aspects of action of GCs in chronic inflammatory diseases, there has been evidence that it is likely that the inhibitory effects of GCs on cytokine synthesis are of particular importance. GCs inhibit the transcription, through the transrepression mechanism, of several cytokines that are relevant in inflammatory diseases, including IL-1β (interleukin-1β), IL-2, IL-3, IL-6, IL-11, TNF-α (tumor necrosis factor-α), GM-CSF (granulocyte-macrophage colony-stimulating factor), and chemokines that attract inflammatory cells to the site of inflammation, including IL-8, RANTES, MCP-1 (monocyte chemotactic protein-1), MCP-3, MCP-4, MIP-1α (macrophage-inflammatory protein-1α), and eotaxin. P. J. Barnes, Clin. Sci., Vol., Vol. 94, 557-572 (1998). On the other hand, there is persuasive evidence that the synthesis of IκB kinases, which are proteins having inhibitory effects on the NF-κB pro-inflammatory transcription factors, is increased by GCs. These pro-inflammatory transcription factors regulate the expression of genes that code for many inflammatory proteins, such as cytokines, inflammatory enzymes, adhesion molecules, and inflammatory receptors. S. Wissink et al., Mol. Endocrinol., Vol. 12, No. 3, 354-363 (1998); P. J. Barnes and M. Karin, New Engl. J. Med., Vol. 336, 1066-1077 (1997). Thus, both the transrepression and transactivation functions of GCs directed to different genes produce the beneficial effect of inflammatory inhibition. On the other hand, steroid-induced diabetes and glaucoma appear to be produced by the transactivation action of GCs on genes responsible for these diseases. H. Schacke et al., Pharmacol. Ther., Vol. 96, 23-43 (2002). Thus, while the transactivation of certain genes by GCs produces beneficial effects, the transactivation of other genes by the same GCs can produce undesired side effects. Therefore, in another aspect, the present invention provides pharmaceutical compositions that avoid generation of one or more adverse side effects of GCs.

In one aspect, an adverse side effect of GCs is selected from the group consisting of glaucoma, cataract, hypertension, hyperglycemia, hyperlipidemia (increased levels of triglycerides), and hypercholesterolemia (increased levels of cholesterol). In one embodiment, a level of said at least an adverse side effect is determined at about one day after said compounds or compositions are first administered to, and are present in, said subject. In another embodiment, a level of said at least an adverse side effect is determined about 30 days after said compounds or compositions are first administered to, and are present in, said subject. Alternatively, a level of said at least an adverse side effect is determined about 2, 3, 4, 5, or 6 months after said compounds or compositions are first administered to, and are present in, said subject.

In another aspect, said at least a prior-art glucocorticoid used to treat or reduce the same condition or disorder is administered to said subject at a dose and a frequency sufficient to produce the same beneficial effect on said condition or disorder as a compound or composition of the present invention after about the same elapsed time.

In still another aspect, said at least a prior-art glucocorticoid is selected from the group consisting of 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, combinations thereof, and mixtures thereof. In one embodiment, said at least a prior-art glucocorticoid is selected from the group consisting of dexamethasone, prednisone, prednisolone, methylprednisolone, medrysone, triamcinolone, loteprednol etabonate, physiologically acceptable salts thereof, combinations thereof, and mixtures thereof. In another embodiment, said at least a prior-art glucocorticoid is acceptable for ophthalmic uses.

In one aspect, a pharmaceutical composition of the present invention comprises an inhibitor of PARP activity, in an amount effective for treating, reducing, ameliorating, alleviating, or preventing a dry eye condition or disorder in a subject.

Non-limiting examples of PARP inhibitors are disclosed below. Other compounds that have been shown to inhibit an activity of PARP also can be used to produce a pharmaceutical composition of the present invention for the treatment, reduction, amelioration, alleviation, or prevention of a dry eye condition or other disorders that require rewetting of the eye.

In one aspect, the PARP inhibitor comprises N-(6-oxo-5,6-dihydrophenanthridin-2-yl)-(N,N-dimethylamino)acetamide hydrochloride, available from Sigma Aldrich. This compound is also known as PJ-34 and has Formula I.

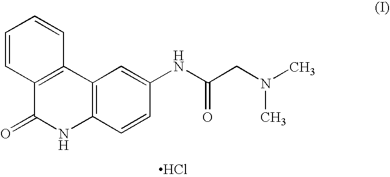

In another aspect, the PARP inhibitor comprises a compound selected from the group consisting of benzamide (having Formula II); 3,4-dihydro-5-hydroxy-1(2H)-isoquinolone (“5OH-DIQ”, having Formula III); 3,4-dihydro-5-[4-1(1-piperidinyl)buthoxy]-1(2H)-isoquinolinone (“DPQ”, having Formula IV); 6(5H)-phenantridinone (“PND”, having Formula V); 3,4-dihydro-5-mercapto-isoquinolin-1(2H)-one (having Formula VI); [3,4-dihydro-5-oxo-isoquinolin-1(2H)-one]-benzoic acid ester (having Formula VII); 3,4-dihydro-5-ethynyl-isoquinolin-1(2H)-one (having Formula VIII); 3,4-dihydro-5-hydroxy-isoquinolin-1(2H)-one (having Formula IX); [3,4-dihydro-5-oxo-isoquinolin-1(2H)-one]-benzoic acid ester (having Formula X); 6-hydroxy-2,3,4,5-tetrahydro-benzo[c]azepin-1-one (having Formula XI); 5-(4-piperidin-1-yl-but-2-ynyloxy)-3,4-dihydro-2H-isoquinolin-1-one (having Formula XII); 5-(5-piperidin-1-yl-pent-1-ynyl)-3,4-dihydro-2H-isoquinolin-1-one (having Formula XIII); 6-(4-piperidin-1-yl-butoxy)-2,3,4,5-tetrahydro-benzo[c]azepin-1-one (having Formula XIV); 2-methyl-8-(4-piperidin-1-yl-butoxy)3H-quinazolin-4-one (having Formula XV); thieno[2,3-c]isoquinolin-5-one (XVI); 9-hydrothieno[2,3-c]isoquinoline-5(4H)-one (having Formula XVII); 9-methoxythieno[2,3-c]isoquinoline-5(4H)-one (having Formula XVIII); and combinations thereof. These compounds are disclosed in A. Chiarugi et al., J. Pharmacol. & Expt'l Therapeutics, Vol. 305, No. 3, 943 (2003), and their methods of preparation are disclosed in R. Pellicciari et al., Il Farmaco, Vol. 58, 851 (2003).

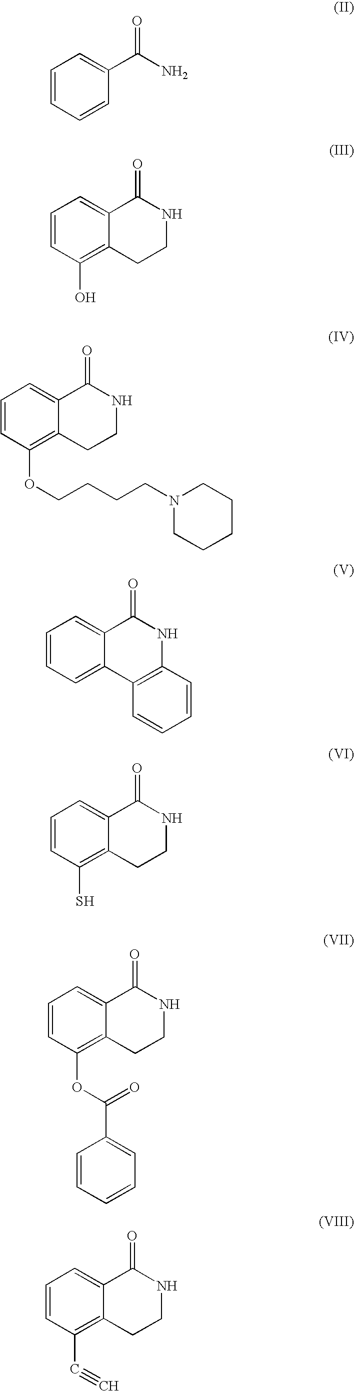

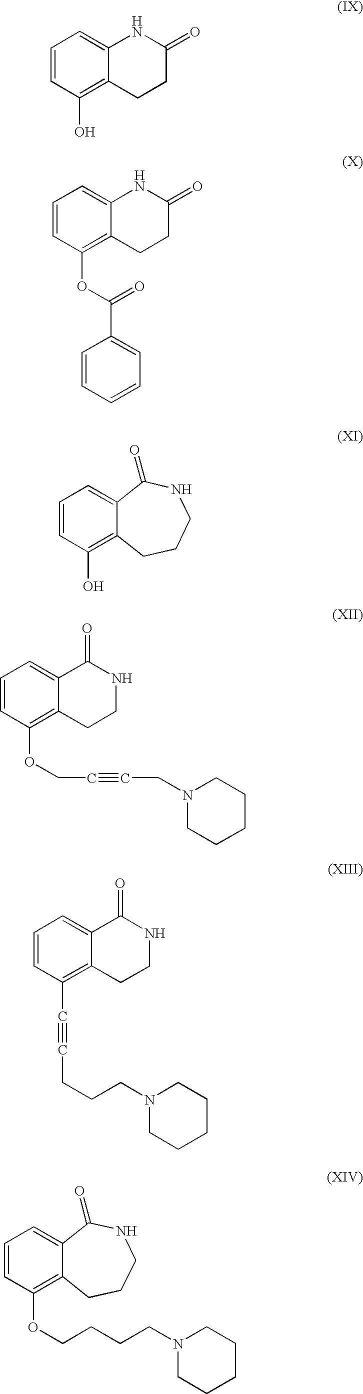

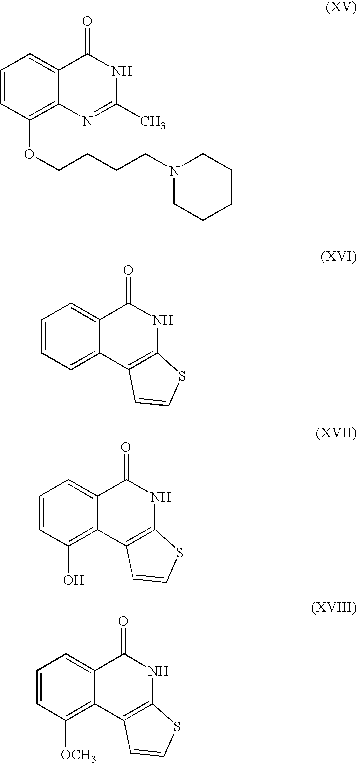

In one embodiment, the PARP inhibitor comprises at least a compound selected from the group consisting of compounds having Formulae I through XVIII.

In another aspect, the PARP inhibitor comprises a propenecarboxylic acid amidoxime derivative disclosed in U.S. Pat. No. 7,151,175, having Formula XIX; an optical isomer; a pharmaceutically suitable acid addition salt; or a quaternary derivative thereof. U.S. Pat. No. 7,151,175 is incorporated herein in its entirety by reference.

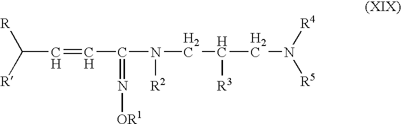

wherein R represents a C₁₋₂₀ alkyl group, a phenyl group, a phenyl group substituted by 1-3 substituents, wherein the substituent is selected from the group consisting of halogen atoms, C₁₋₃ alkyl groups, C₁₋₃ alkoxy groups, an amino group, (C₁₋₄ alkyl)amino groups, di(C₁₋₄ alkyl)amino groups, (C₁₋₄ alkanoyl)amino groups, and 5- or 6-membered saturated or unsaturated heterocyclic groups containing one or two nitrogen atoms or a sulphur atom as the heteroatom and each of said heterocyclic groups is optionally fused with one or more benzene rings, or one or more heterocyclic groups or one or more benzene rings and one or more heterocyclic groups; and R′ represents a hydrogen atom; or R forms together with R′ a C₅₋₇ cycloalkyl group optionally fused with a benzene ring; R⁴ and R⁵ represent, independently, a hydrogen atom, a C₁₋₅ alkyl group, a C₁₋₅ alkanoyl group, a phenyl group, a phenyl group substituted by 1-3 substituents, wherein the substituent is selected from the group consisting of halogen atoms, C₁₋₃ alkyl groups, and C₁₋₃ alkoxy groups; or R⁴ and R⁵ form together with the adjacent nitrogen atom a 5- or 6-membered saturated or unsaturated heterocyclic group that contains no or one additional heteroatom, wherein the additional heteroatom is selected from the group consisting of nitrogen, oxygen, and sulphur as the heteroatom, and that can be fused with a benzene ring, and each of the heterocyclic group and the benzene ring bears no, one, or two substituents, wherein the substituent is selected from the group consisting of halogen atoms, C₁₋₂ alkyl groups, C₁₋₂ alkoxy groups; and R¹, R², and R³ satisfy one of the following conditions: (i) R¹ and R² represent a hydrogen atom, R³ represents a hydrogen atom, a hydroxy group, or a C₁₋₅ alkoxy group; (ii) R¹ forms together with R² a carbonyl group or a thiocarbonyl group, the carbon atom of which is bound to the oxygen atom adjacent to R¹ and to the nitrogen atom adjacent to R², and R³ represents a hydrogen atom, a halogen atom, a hydroxy group, a C₁₋₅ alkoxy group, a C₁₋₅ alkylthio group, a C₁₋₂₀ alkanoyloxy group, a C₃₋₂₂ alkenoyloxy group containing one or more double bonds, a methylsulfonyloxy group, a benzenesulfonyloxy group, or a toluenesulfonyloxy group; or (iii) R² is a hydrogen atom and R¹ forms together with R³ a valence bond between the oxygen atom adjacent to R¹ and the carbon atom adjacent to R³.

Non-limiting examples of the compounds having Formula XIX include 3-styryl-4-(3-piperidino-2-hydroxpropyl)-Δ²-1,2,4-oxadiazolin-5-one; 3-styryl-4-(3-pyrrolidino-2-hydroxproxypropyl)-Δ²-1,2,4-oxadia-zolin-5-one; 3-styryl-4-(3-hexamethyleneimino-2-hydroxypropyl)-Δ²-1,2,4-oxa-diazolin-5-one; 3-styryl-4-(3-morpholino-2-hydroxypropyl)-Δ²-1,2,4-oxadiazolin-5-one; 3-styryl-4-[3-(tert.-butylamino)-2-hydroxypropyl]-Δ²-1,2,4-oxadiazolin-5-one; 3-styryl-4-(3-(1,2,3,4-tetrahydro-2-isoquinoyl)-2-hydroxypropyl-Δ²-1,2,4-oxadiazolin-5-one; 3-styryl-4-[3-(2,6-dimethylanilino)-2-hydroxypropyl]-Δ²-1,2,4-oxadiazolin-5-one; and 3-(3,4-dimethoxystyrl-4-(3-piperidino-2-hydroxypropyl)-Δ²-1,2,4-oxadiazolin-5-one.

In another aspect, the PARP inhibitor comprises a substituted indole disclosed in U.S. Pat. No. 7,087,637, which is incorporated herein in its entirety by reference. Non-limiting examples of such a substituted indole include 2-(4(4-n-propylpiperazin-1-yl)phenyl)-1H-indole-4-carboxamide; 2-(4-piperazin-1-ylphenyl)-1H-indole-4-carboxamide; 2-(4(4-isopropylpiperazin-1-yl)phenyl)-1H-indole-4-carboxamide; 2-(4(4-benzylpiperazin-1-yl)phenyl)-1H-indole-4-carboxamide; 2-(4(4-n-butylpiperazin-1-yl)phenyl)-1H-indole-4-carboxamide; 2-(4(4-ethylpiperazin-1-yl)phenyl)-1H-indole-4-carboxamide; 2-(4-(2-N,N-dimethylaminoeth-1-yloxy)phenyl)-1H-indole-4-carboxamide; 2-(4-(2-pyrrolidin-1-yleth-1-yloxy)phenyl)-1H-indole-4-carboxamide; 2-(4-(2-piperidm-1-yleth-1-yloxy)phenyl)-1H-indole-4-carboxamide; 2-(4-(2-piperazin-1-yleth-1-yloxy)phenyl)-1H-indole-4-carboxamide; 2-(4-(2-(4-methylpiperazin-1-yl)eth-1-yloxy)phenyl)-1H-indole-4-carboxamide; 2-(4-(2-(4-propylpiperazin-1-yl)eth-1-yloxy)phenyl)-1H-indole-4-carboxamide; 2-(4-(2-(4-ethylpiperazin-1-yl)eth-1-yloxy)phenyl)-1H-indole-4-carboxamide; 2-(4-(2-(4-benzylpiperazin-1-yl)eth-1-yloxy)phenyl)-1H-indole-4-carboxamide; 2-(4-(2-(4-acetamidopiperazin-1-yl)eth-1-yloxy)phenyl)-1H-indole-4-carboxamide; 2-(4-(2-(4-benzanidopiperazin-1-yl)eth-1-yloxy)phenyl)-1H-indole-4-carboxamide; 2-(4-homopiperazin-1-ylphenyl)-1H-indole-4-carboxamide; 2-(4(4-methylhomopiperazin-1-yl)phenyl)-1H-indole-4-carboxamide; 2-(4(4-benzylhomopiperazin-1-yl)phenyl)-1H-indole-4-carboxamide; 2-(4-(4-n-butylhomopiperazin-1-yl)phenyl)-1H-indole-4-carboxamide; 2-(4(4-ethylhomopiperazin-1-yl)phenyl)-1H-indole-4-carboxamide; 2-(4-methoxyphenyl)-1H-indole-4-carboxamide; 2-(4-chlorophenyl)-1H-indole-4-carboxamide; 2-(4-aminophenyl)-1H-indole-4-carboxamide; 2-(4-methylphenyl)-1H-indole-4-carboxamide; 2-(4-phenylphenyl)-1H-indole-4-carboxamide; 2-(4-isopropylphenyl)-1H-indole-4-carboxamide; 2-(4-fluorophenyl)-1H-indole-4-carboxamide; 2-(4-trifluoromethylphenyl)-1H-indole-4-carboxamide; 2-(3-methoxyphenyl)-1H-indole-4-carboxamide; 2-(3-chlorophenyl)-1H-indole-4-carboxamide; 2-(3-aminophenyl)-1H-indole-4-carboxamide; 2-(3-methylphenyl)-1H-indole-4-carboxamide; 2-(3-phenylphenyl)-1H-indole-4-carboxamide; 2-(3-isopropylphenyl)-1H-indole-4-carboxamide; 2-(3-fluorophenyl)-1H-indole-4-carboxamide; 2-(3-trifluoromethylphenyl)-1H-indole-4-carboxamide; 2-piperidin-4-yl-1H-indole-4-carboxamide; 2-(1-methylpiperidin-4-yl)-1H-indole-4-carboxamide; 2-(1-n-propylpiperidin-4-yl)-1H-indole-4-carboxamide; 2-(1-benzylpiperidin-4-yl)-1H-indole-4-carboxamide; 2-(1-n-butylpiperidin-4-yl)-1H-indole-4-carboxamide; 2-(1-isopropylpiperidin-4-yl)-1H-indole-4-carboxamide; 2-pyridin-4-yl-1H-indole-4-carboxamide; 2-pyridin-3-yl-1H-indole-4-carboxamide; 2-pyridin-2-yl-1H-indole-4-carboxamide; 2-thien-2-yl-1H-indole-4-carboxamide; 2-thien-3-yl-1H-indole-4-carboxamide; 2-indol-3-yl-1H-indole-4-carboxamide; 2-indol-5-yl-1H-indole-4-carboxamide; 2-indol-2-yl-1H-indole-4-carboxamide; 2-quinolin-3-yl-1H-indole-4-carboxamide; 2-quinolin-2-yl-1H-indole-4-carboxamide; 2-quinolin-4-yl-1H-indole-4-carboxamide; 2-isoquinolin-1-yl-1H-indole-4-carboxamide; 2-isoquinolin-3-yl-1H-indole-4-carboxamide; 2-quinoxalin-2-yl-1H-indole-4-carboxamide; 2-naphth-2-yl-1H-indole-4-carboxamide; 2-naphth-1-yl-1H-indole-4-carboxamide; 2-(2(N,N-dimethylamino)eth-1-ylamino)phenyl)-1H-indole-4-carboxamide; 2-(2(N,N-diethylamino)eth-1-ylamino)phenyl)-1H-indole-4-carboxamide; 2-(2-piperidin-1-yleth-1-ylamino)phenyl)-1H-indole-4-carboxamide; 2-(2-pyrrolidin-1-yleth-1-ylamino)phenyl)-1H-indole-4-carboxamide; 2-(3(N,N-dimethylamino)prop-1-ylamino)phenyl)-1H-indole-4-carboxamide; 2-(3(N,N-diethylamino)prop-1-ylamino)phenyl)-1H-indole-4-carboxamide; 2-(3-piperidin-1-ylprop-1-ylamino)phenyl)-1H-indole-4-carboxamide; 2-(3-pyrrolidin-1-ylprop-1-ylamino)phenyl)-1H-indole-4-carboxamide; 2-cyclohexyl-1H-indole-4-carboxamide; 2-(cis-4-aminocyclohex-1-yl)-1H-indole-4-carboxamide; 2-(4-methoxycyclohex-1-yl)-1H-indole-4-carboxamide; 2-(4(4-n-propylpiperazin-1-yl)phenyl)-1,3,4,5-tetrahydro-6H-azepino[5,4,3-c,d]indol-6-one; 2-(4-piperazin-1-ylphenyl)-1,3,4,5-tetrahydro-6H-azepino[5,4,3-c,d]indol-6-one; 2-(4(4-isopropylpiperazin-1-yl)penyl)-1,3,4,5-tetrahydro-6H-azepino[5,4,3-c,d]indol-6-one; 2-(4(4-benzylpiperazin-1-yl)phenyl)-1,3,4,5-tetrahydro-6H-azepino[5,4,3-c,d]indol-6-one; 2-(4(4-n-butylpiperazin-1-yl)phenyl)-1,3,4,5-tetrahydro-6H-azepino[5,4,3-c,d]indol-6-one; 2-(4(4-ethylpiperazin-1-yl)phenyl)-1,3,4,5-tetrahydro-6H-azepino[5,4,3-c,d]indol-6-one; 2-(4-(2-N,N-dimethylaminoeth-1-yloxy)phenyl)-1,3,4,5-tetrahydro-6H-azepino[5,4,3-c,d]indol-6-one; 2-(4-(2-pyrrolidin-1-yleth-1-yloxy)phenyl)-1,3,4,5-tetrahydro-6H-azepino[5,4,3-c,d]indol-6-one; 2-(4-(2-piperidin-1-yleth-1-yloxy)phenyl)-1,3,4,5-tetrahydro-6H-azepino[5,4,3-c,d]indol-6-one; 2-(4-(2-piperazin-1-yleth-1-yloxy)phenyl)-1,3,4,5-tetrahydro-6H-azepino[5,4,3-c,d]indol-6-one; 2-(4-(2-(4-methylpiperazin-1-yl)eth-1-yloxy)phenyl)-1,3,4,5-tetrahydro-6H-azepino[5,4,3-c,d]indol-6-one; 2-(4-(2-(4-propylpiperazin-1-yl)eth-1-yloxy)phenyl)-1,3,4,5-tetrahydro-6H-azepino[5,4,3-c,d]indol-6-one; 2-(4-(2-(4-ethylpiperazin-1-yl)eth-1-yloxy)phenyl)-1,3,4,5-tetrahydro-6H-azepino[5,4,3-c,d]indol-6-one; 2-(4-(2-(4-benzylpiperazin-1-yl)eth-1-yloxy)phenyl)-1,3,4,5-tetrahydro-6H-azepino[5,4,3-c,d]indol-6-one; 2-(4-(2-(4-acetamidopiperazin-1-yl)eth-1-yloxy)phenyl)-1,3,4,5-tetrahydro-6H-azepino[5,4,3-c,d]indol-6-one; 2-(4-(2-(4-benzamidopiperazin-2-yl)eth-1-yloxy)phenyl)-1,3,4,5-tetrahydro-6H-azepino[5,4,3-c,d]indol-6-one; 2-(4-homopiperazin-1-ylphenyl)-1,3,4,5-tetrahydro-6H-azepino[5,4,3-c,d]indol-6-one; 2-(4(4-Mmthylhomopiperazin-1-yl)phenyl)-1,3,4,5-tetrahydro-6H-azepino[5,4,3-c,d]indol-6-one; 2-(4(4-benzylhomopiperazin-1-yl)phenyl)-1,3,4,5-tetrahydro-6H-azepino[5,4,3-c,d]indol-6-one; 2-(4-(4-n-butylhomopiperazin-1-yl)phenyl)-1,3,4,5-tetrahydro-6H-azepino[5,4,3-c,d]indol-6-one; 2-(4(4-ethylhomopiperazin-1-yl)phenyl)-1,3,4,5-tetrahydro-6H-azepino[5,4,3-c,d]indol-6-one; 2-piperidin-4-yl-1,3,4,5-tetrahydro-6H-azepino[5,4,3-c,d]indol-6-one; 2-(1-methylpiperidin-4-yl)-1,3,4,5-tetrahydro-6H-azepino[5,4,3-c,d]indol-6-one; 2-(1-n-propylpiperidin-4-yl)-1,3,4,5-tetrahydro-6H-azepino[5,4,3-c,d]indol-6-one; 2-(1-benzylpiperidin-4-yl)-1,3,4,5-tetrahydro-6H-azepino[5,4,3-c,d]indol-6-one; 2-(1-n-butylpiperidin-4-yl)-1,3,4,5-tetrahydro-6H-azepino[5,4,3-c,d]indol-6-one; 2-(1-isopropylpiperidin-4-yl)-1,3,4,5-tetrahydro-6H-azepino[5,4,3-c,d]indol-6-one; 2-pyridin-4-yl-1,3,4,5-tetrahydro-6H-azepino[5,4,3-c,d]indol-6-one; 2-pyridin-3-yl-1,3,4,5-tetrahydro-6H-azepino[5,4,3-c,d]indol-6-one; 2-pyridin-2-yl-1,3,4,5-tetrahydro-6H-azepino[5,4,3-c,d]indol-6-one; 2-thien-2-yl-1,3,4,5-tetrahydro-6H-azepino[5,4,3-c,d]indol-6-one; 2-thien-3-yl-1,3,4,5-tetrahydro-6H-azepino[5,4,3-c,d]indol-6-one; 2-indol-3-yl-1,3,4,5-tetrahydro-6H-azepino[5,4,3-c,d]indol-6-one; 2-indol-5-yl-1,3,4,5-tetrahydro-6H-azepino[5,4,3-c,d]indol-6-one; 2-indol-2-yl-1,3,4,5-tetrahydro-6H-azepino[5,4,3-c,d]indol-6-one; 2-quinolin-3-yl-1,3,4,5-tetrahydro-6H-azepino[5,4,3-c,d]indol-6-one; 2-quinolin-2-yl-1,3,4,5-tetrahydro-6H-azepino[5,4,3-c,d]indol-6-one; 2-quinolin-4-yl-1,3,4,5-tetrahydro-6H-azepino[5,4,3-c,d]indol-6-one; 2-isoquinolin-1-yl-1,3,4,5-tetrahydro-6H-azepino[5,4,3-c,d]indol-6-one; 2-isoquinolin-3-yl-1,3,4,5-tetrahydro-6H-azepino[5,4,3-c,d]indol-6-one; 2-quinoxalin-2-yl-1,3,4,5-tetrahydro-6H-azepino[5,4,3-c,d]indol-6-one; 2-naphth-2-yl-1,3,4,5-tetrahydro-6H-azepino[5,4,3-c,d]indol-6-one; 2-naphth-1-yl-1,3,4,5-tetrahydro-6H-azepino[5,4,3-c,d]indol-6-one; 2-(2(N,N-dimethylamino)eth-1-ylamino)phenyl)-1,3,4,5-tetrahydro-6H-azepino[5,4,3-c,d]indol-6-one; 2-(2(N,N-diethylamino)eth-1-ylamino)phenyl)-1,3,4,5-tetrahydro-6H-azepino[5,4,3-c,d]indol-6-one; 2-(2-piperidin-1-yleth-1-ylamino)phenyl)-1,3,4,5-tetrahydro-6H-azepino[5,4,3-c,d]indol-6-one; 2-(2-pyrrolidin-1-yleth-1-ylamino)phenyl)-1,3,4,5-tetrahydro-6H-azepino[5,4,3-c,d]indol-6-one; 2-(3 (N,N-dimethylamino)prop-1-ylamino)phenyl)-1,3,4,5-tetrahydro-6H-azepino[5,4,3-c,d]indol-6-one; 2-(3 (N,N-diethylamino)prop-1-ylamino)phenyl)-1,3,4,5-tetrahydro-6H-azepino[5,4,3-c,d]indol-6-one; 2-(3-piperidin-1-ylprop-1-ylamino)phenyl)-1,3,4,5-tetrahydro-6H-azepino[5,4,3-c,d]indol-6-one; 2-(3-pyrrolidin-1-ylprop-1-ylamino)phenyl)-1,3,4,5-tetrahydro-6H-azepino[5,4,3-c,d]indol-6-one; 2-cyclohexyl-1,3,4,5-tetrahydro-6H-azepino[5,4,3-c,d]indol-6-one; 2-(cis-4-aminocyclohex-1-yl)-1,3,4,5-tetrahydro-6H-azepino[5,4,3-c,d]indo-1-6-one; and 2-(4-methoxycyclohex-1-yl)-1,3,4,5-tetrahydro-6H-azepino[5,4,3-c,d]indol-6-one.

In still another aspect, the PARP inhibitor comprises an imidazopyridine derivative, as disclosed in U.S. Pat. No. 7,041,675, which is incorporated herein in its entirety by reference. Non-limiting examples of such an imidazopyridine derivative include 2-(4-(4-n-propyl-piperazin-1-yl)-phenyl)-imidazo[1,2-a]pyridine-8-carboxamide; 2-(4-piperazin-1-yl-phenyl)-imidazo[1,2-a]pyridine-8-carboxamide; 2-(4-(4-isopropyl-piperazin-1-yl)-phenyl)-imidazo[1,2-a]pyridine-8-carboxamide; 2-(4-(4-benzylpiperazin-1-yl)-phenyl)-imidazo[1,2-a]pyridine-8-carboxamide; 2-(4-(4-n-butyl-piperazin-1-yl)-phenyl)-imidazo[1,2-a]pyridine-8-carboxamide; 2-(4-(4-ethyl-piperazin-1-yl)-phenyl)-imidazo[1,2-a]pyridine-8-carboxamide; 2-(4-(2-N,N-dimethylaminoeth-1-yloxy)-phenyl)-imidazo-[1,2-a]pyridine-8-carboxamide; 2-(4-(2-pyrrolidin-1-yl-eth-1-yloxy)-phenyl)-imidazo[1,2-a]pyridine-8-carboxamide; 2-(4-(2-piperidin-1-yl-eth-1-yloxy)-phenyl)-imidazo[1,2-a]pyridine-8-carboxamide; 2-(4-(2-piperazin-1-yl-eth-1-yloxy)-phenyl)-imidazo[1,2-a]pyridine-8-carboxamide; 2-(4-(2-(4-methyl-piperazin-1-yl)-eth-1-yloxy)-phenyl)-imidazo[1,2-a]pyridine-8-carboxamide; 2-(4-(2-(4-propyl-piperazin-1-yl)-eth-1-yloxy)-phenyl-imidazo[1,2-a]pyridine-8-carboxamide; 2-(4-(2-(4-ethyl-piperazin-1-yl)-eth-1-yloxy)-phenyl-imidazo[1,2-a]pyridine-8-carboxamide; 2-(4-(2-(4-benzyl-piperazin-1-yl)-eth-1-yloxy)-phenyl-imidazo[1,2-a]pyridine-8-carboxamide; 2-(4-(2-(4-acetamido-piperazin-1-yl)-eth-1-yloxy)-phenyl-imidazo[1,2-a]pyridine-8-carboxamide; 2-(4-(2-(4-benzamido-piperazin-1-yl)-eth-1-yloxy)-phenyl-imidazo[1,2-a]pyridine-8-carboxamide; 2-(4-homopiperazin-1-yl-phenyl)-imidazo[1,2-a]pyridine-8-carboxamide; 2-(4(4-methylhomopiperazin-1-yl)-phenyl)-imidazo[1,2-a]pyridine-8-carboxamide; 2-(4(4-benzylhomopiperazin-1-yl)-phenyl)-imidazo[1,2-a]pyridine-8-carboxamide; 2-(4-(4-n-butyl-homopiperazin-1-yl)-phenyl)-imidazo[1,2-a]pyridine-8-carboxamide; 2-(4(4-ethylhomo-piperazin-1-yl)-phenyl)-imidazo[1,2-a]pyridine-8-carboxamide; 2-(4-methoxy-phenyl)-imidazo[1,2-a]pyridine-8-carboxamide; 2-(4-methyl-phenyl)-imidazo[1,2-a]pyridine-8-carboxamide; 2-(4-phenyl-phenyl)-imidazo[1,2-a]pyridine-8-carboxamide; 2-(4-isopropyl-phenyl)-imidazo[1,2-a]pyridine-8-carboxamide; 2-(4-fluoro-phenyl)-imidazo[1,2-a]pyridine-8-carboxamide; 2-(4-trifluoromethyl-phenyl)-imidazo[1,2-a]pyridine-8-carboxamide; 2-(3-methoxy-phenyl)-imidazo[1,2-a]pyridine-8-carboxamide; 2-(3-chloro-phenyl)-imidazo[1,2-a]pyridine-8-carboxamide; 2-(3-amino-phenyl)-imidazo[1,2-a]pyridine-8-carboxamide; 2-(3-methyl-phenyl)-imidazo[1,2-a]pyridine-8-carboxamide; 2-(3-phenyl-phenyl)-imidazo[1,2-a]pyridine-8-carboxamide; 2-(3-isopropyl-phenyl)-imidazo[1,2-a]pyridine-8-carboxamide; 2-(3-fluoro-phenyl)-imidazo[1,2-a]pyridine-8-carboxamide; 2-(3-trifluoromethyl-phenyl)-imidazo[1,2-a]pyridine-8-carboxamide; 2-piperidin-4-yl-imidazo[1,2-a]pyridine-8-carboxamide; 2-(1-methyl-piperidin-4-yl)-imidazo[1,2-a]pyridine-8-carboxamide; 2-(1-ethyl-piperidin-4-yl)-imidazo[1,2-a]pyridine-8-carboxamide; 2-(1-n-propyl-piperidin-4-yl)-imidazo[1,2-a]pyridine-8-carboxamide; 2-(1-benzyl-piperidin-4-yl)-imidazo[1,2-a]pyridine-8-carboxamide; 2-(1-n-butyl-piperidin-4-yl)-imidazo[1,2-a]pyridine-8-carboxamide; 2-(1-isopropyl-piperidin-4-yl)-imidazo[1,2-a]pyridine-8-carboxamide; 2-pyridin-4-yl-imidazo[1,2-a]pyridine-8-carboxamide; 2-pyridin-3-yl-imidazo[1,2-a]pyridine-8-carboxamide; 2-pyridin-2-yl-imidazo[1,2-a]pyridine-8-carboxamide; 2-thien-2-yl-imidazo[1,2-a]pyridine-8-carboxamide; 2-thien-3-yl-imidazo[1,2-a]pyridine-8-carboxamide; 2-indol-3-yl-imidazo[1,2-a]pyridine-8-carboxamide; 2-indol-5-yl-imidazo[1,2-a]pyridine-8-carboxamide; 2-indol-2-yl-imidazo[1,2-a]pyridine-8-carboxamide; 2-quinolin-3-yl-imidazo[1,2-a]pyridine-8-carboxamide; 2-quinolin-2-yl-imidazo[1,2-a]pyridine-8-carboxamide; 2-quinolin-4-yl-imidazo[1,2-a]pyridine-8-carboxamide; 2-isoquinolin-1-yl-imidazo[1,2-a]pyridine-8-carboxamide; 2-isoquinolin-3-yl-imidazo[1,2-a]pyridine-8-carboxamide; 2-quinoxalin-2-yl-imidazo[1,2-a]pyridine-8-carboxamide; 2-naphth-2-yl-imidazo[1,2-a]pyridine-8-carboxamide; 2-naphth-1-yl-imidazo[1,2-a]pyridine-8-carboxamide; 2-(4-(2(N,N-dimethylamino)-eth-1-ylamino)-phenyl)-imidazo[1,2-a]pyridine-8-carboxamide; 2-(4-(2(N,N-diethylamino)-eth-1-ylamino)-phenyl)-imidazo[1,2-a]pyridine-8-carboxamide; 2-(4-(2-piperidin-1-yl-eth-1-ylamino)-phenyl)-imidazo[1,2-a]pyridine-8-carboxamide; 2-(4-(2-pyrrolidin-1-yl-eth-1-ylamino)-phenyl)-imidazo[1,2-a]pyridine-8-carboxamide; 2-(4-(3 (N,N-dimethylamino)-prop-1-ylamino)-phenyl)-imidazo[1,2-a]pyridine-8-carboxamide; 2-(4-(3 (N,N-diethylamino)-prop-1-ylamino)-phenyl)-imidazo[1,2-a]pyridine-8-carboxamide; 2-(4-(3-piperidin-1-yl-prop-1-ylamino)-phenyl)-imidazo[1,2-a]pyridine-8-carboxamide; 2-(4-(3-pyrrolidin-1-yl-prop-1-ylamino)-phenyl)-imidazo-[1,2-a]pyridine-8-carboxamide; 2-cyclohexyl-imidazo[1,2-a]pyridine-8-carboxamide; 2-(cis-4-amino-cyclohex-1-yl)-imidazo[1,2-a]pyridine-8-carboxamide; 2-(4-methoxy-cyclohex-1-yl)-imidazo[1,2-a]pyridine-8-carboxamide; 2-(4-(2(N,N-dimethylamino)-eth-1-yl-methylamino)-phenyl)-imidazo[1,2-a]-pyridine-8-carboxamide; and 2-(4-(4-methyl-piperazin-1-yl)-phenyl)-imidazo[1,2-a]pyridine-8-carboxamide.

In still another aspect, the PARP inhibitor comprises one of the fused tricyclic compounds, as disclosed in U.S. Pat. Nos. 6,548,494 and 6,977,298, which are incorporated herein in their entirety by reference. In certain embodiments, such fused tricyclic compounds include pyrroloisoquinoline derivatives, azepinoindole derivatives, and triazabenzoazulene derivatives. Non-limiting examples of such fused tricyclic compounds include 3,4-dihydropyrrolo[4,3,2-de]isoquinolin-5-(1H)-one; 2-bromo-3,4-dihydropyrrolo[4,3,2-de]isoquinolin-5-(1H)-one; phenyl-3,4-dihydropyrrolo[4,3,2-de]isoquinolin-5-(1H)-one; 3,4,5,6-tetrahydro-1H-azepino[5,4,3-cd]indol-6-one; 2-bromo-3,4,5,6-tetrahydro-1H-azepino[5,4,3-cd]indol-6-one; 2-phenyl-3,4,5,6-tetrahydro-1H-azepino[5,4,3-cd]indol-6-one; 2-(4-Methoxyphenyl)-3,4,5,6-tetrahydro-1H-azepino[5,4,3-cd]indol-6-one; 2-(3-nitrophenyl)-3,4,5,6-tetrahydro-1H-azepino[5,4,3-cd]indol-6-one; 2-(3-hydroxymethylphenyl)-3,4,5,6-tetrahydro-1H-azepino[5,4,3-cd]indol-6-one; 2-(phenylethynyl)-3,4,5,6-tetrahydro-1H-azepino[5,4,3-cd]indol-6-one; 1-methyl-2-phenyl-3,4,5,6-tetrahydro-1H-azepino[5,4,3-cd]indol-6-one; 1-N-methyl-3,4,5,6-tetrahydro-1H-azepino[5,4,3-cd]indol-6-one; (rac)-3-phenyl-3,4,5,6-tetrahydro-1H-azepino[5,4,3-cd]indol-6-one; 2-(4-fluorophenyl)-3,4,5,6-tetrahydro-1H-azepino[5,4,3-cd]indol-6-one; 8-bromo-2-phenyl-3,4,5,6-tetrahydro-1H-azepino[5,4,3-cd]indol-6-one; 2-(4-(N,N-dimethylamino)methylphenyl)-3,4,5,6-tetrahydro-1H-azepino[5,4,3-c d]indol-6-one; 2-(3-(N,N-dimethylamino)methylphenyl)-3,4,5,6-tetrahydro-1H-azepino[5,4,3-c d]indol-6-one; 1,5-dihydro-[1,2]diazepino[4,5,6-cd]-indol-6-one; 1,5-dihydro-3-phenyl-[1,2]diazepino[4,5,6-cd]-indol-6-one; 1,5-dihydro-3-phenethyl-[1,2]diazepino[4,5,6-cd]-indol-6-one; 2-(3-trifluoromethyl-phenyl)-1,3,4,5-tetrahydro-azepino[5,4,3-cd]indol-6-one; 2-(4-trifluoromethyl-phenyl)-1,3,4,5-tetrahydro-azepino[5,4,3-cd]indol-6-one; 2-benzofuran-2-yl-1,3,4,5-tetrahydro-azepino[5,4,3-cd]indol-6-one; 2-(3,5-bis-trifluoromethyl-phenyl)-1,3,4,5-tetrahydro-azepino[5,4,3-cd]indo 1-6-one; 2-(4-bromophenyl)-1,3,4,5-tetrahydro-azepino[5,4,3-cd]indol-6-one; 2-(3-chloro-4-fluoro-phenyl)-1,3,4,5-tetrahydro-azepino[5,4,3-cd]indol-6-one; 2-(4-tert-butyl-phenyl)-1,3,4,5-tetrahydro-azepino[5,4,3-cd]indol-6-one; 2-phenyl-1,3,4,5-tetrahydro-azepino[5,4,3-cd]indole-6-thione; 2-phenethyl-1,3,4,5-tetrahydro-azepino[5,4,3-cd]indol-6-one; 2-(2-chlorophenyl)-1,3,4,5-tetrahydro-azepino[5,4,3-cd]indol-6-one; 2-naphthalen-1-yl-1,3,4,5-tetrahydro-azepino[5,4,3-cd]indol-6-one; 6-oxo-3,4,5,6-tetrahydro-1H-azepino[5,4,3-cd]indole-2-carboxylic acid methyl ester; 2-iodo-1,3,4,5-tetrahydro-azepino[5,4,3-cd]indol-6-one; 2-(4-(N-methylamino)methylphenyl)-3,4,5,6-tetrahydro-1H-azepino[5,4,3-cd]indol-6-one; 2-(3-(N-methylamino)methylphenyl)-3,4,5,6-tetrahydro-1H-azepino[5,4,3-cd]indol-6-one; 2-(3-(3-piperidin-1-ylmethylphenyl)-3,4,5,6-tetrahydro-1H-azepino[5,4,3-cd]indol-6-one; N-[3-(6-oxo-3,4,5,6-tetrahydro-1H-azepino[5,4,3-cd]indol-2-yl)-phenyl]-acetamide; 3-[2-(6-oxo-3,4,5,6-tetrahydro-1H-azepino[5,4,3-cd]indol-2-yl)-phenyl]-propionic acid methyl ester; 2-pyridin-3-yl-1,3,4,5-tetrahydro-azepino[5,4,3-cd]indol-6-one; 2-(2-methylsulfanyl-phenyl)-1,3,4,5-tetrahydro-azepino[5,4,3-cd]indol-6-one; 2-[4-(2-pyrrolidin-1-yl-ethyl)-phenyl]-1,3,4,5-tetrahydro-azepino[5,4,3-cd]indol-6-one; N-[4-fluoro-2-(6-oxo-3,4,5,6-tetrahydro-1H-azepino[5,4,3-cd]indol-2-yl)-phenyl]-acetamide; 6-oxo-3,4,5,6-tetrahydro-1H-azepino[5,4,3-cd]indole-2-carboxylic acid methylamide; 4-(6-oxo-3,4,5,6-tetrahydro-1H-azepino[5,4,3-cd]indol-2-yl)-benzoic acid; 6-oxo-3,4,5,6-tetrahydro-1H-azepino[5,4,3-cd]indole-2-carboxylic acid; 6-oxo-3,4,5,6-tetrahydro-1H-azepino[5,4,3-cd]indole-2-carboxylic acid (4-fluoro-phenyl)-amide; 2-(1H-pyrrol-2-yl)-1,3,4,5-tetrahydro-azepino[5,4,3-cd]indol-6-one; (RS)-(.+−.)-1-(4-chloro-phenyl)-9-(4-methoxy-phenyl)-8,9-dihydro-7H-2,7,9a-triaza-benzo[cd]azulen-6-one; (3-[1,3]dioxolan-2-yl-phenyl)-8,9-dihydro-7H-2,7,9a-triaza-benzo[cd]azulen-6-one; 1-(4-diethoxymethyl-phenyl)-8,9-dihydro-7H-2,7,9a-triaza-benzo[cd]azulen-6-one; 1-(3-pyrrolidin-1-ylmethyl-phenyl)-8,9-dihydro-7H-2,7,9a-triaza-benzo[cd]azulen-6-one; 1-[3-(3-trifluoromethyl-phenoxy)-phenyl]-8,9-dihydro-7H-2,7,9a-triaza-benzo[cd]azulen-6-one; 1-(3-[1,3]dioxolan-2-yl-phenyl)-4-fluoro-8,9-dihydro-7H-2,7,9a-triaza-benzo [cd]azulen-6-one; 1-(3-dimethylaminomethyl-phenyl)-4-fluoro-8,9-dihydro-7H-2,7,9a-triaza-benzo[cd]azulen-6-one; 1-(4-morpholin-4-ylmethyl-phenyl)-8,9-dihydro-7H-2,7,9a-triaza-benzo[cd]azulen-6-one; 1-(3-p-tolyl-benzo[c]isoxazol-5-yl)-8,9-dihydro-7H-2,7,9a-triaza-benzo[cd]azulen-6-one; 4-[5-(6-oxo-6,7,8,9-tetrahydro-2,7,9a-triaza-benzo[cd]azulen-1-yl)-pyridin-2-yloxy]-benzonitrile; 6-oxo-6,7,8,9-tetrahydro-2,7,9a-triaza-benzo[cd]azulen-1-carboxylic acid benzylamide; 1-(4-methyl-piperazine-1-carbonyl)-8,9-dihydro-7H-2,7,9a-triaza-benzo[cd]azulen-6-one; 1-[4-(2-pyrrolidin-1-yl-ethyl)-phenyl]-8,9-dihydro-7H-2,7,9a-triaza-benzo[c d]azulen-6-one; 1-[4-((2S,5S)-2,5-bis-methoxymethyl-pyrrolidin-1-ylmethyl)-phenyl]-8,9-dihydro-7H-2,7,9a-triaza-benzo[cd]azulen-6-one; and (R)-1-(4-dimethylaminomethyl-phenyl)-8-hydroxymethyl-8,9-dihydro-7H-2,7,9a-triaza-benzo[cd]azulen-6-one.

In still another aspect, the PARP inhibitor comprises a phthalazinone derivative, a pyrazinopyridazinone derivative, or a pyridinopyridazinone derivative, as disclosed in U.S. Pat. No. 6,924,284, which is incorporated herein in its entirety by reference. Non-limiting examples of such compounds include N-[3-(4-oxo-3,4-dihydro-phthalazin-1-ylamino)-propyl]-3-[3-(1H-pyrrol-2-yl)-[1,2,4]oxadiazol-5-yl]-propionamide; N-[3-(4-oxo-3,4-dihydro-phthalazin-1-ylamino)-propyl]-3-(3-thiophen-3-yl-[1,2,4]oxadiazol-5-yl)-propionamide; 3-[3-(2-methyl-thiophen-3-yl)-[1,2,4]oxadiazol-5-yl]-N-[3-(4-oxo-3,4-dihydro-phthalazin-1-ylamino)-propyl]-propionamide; 3-[3-(4-methanesulfonylamino-phenyl)-[1,2,4]oxadiazol-5-yl]-N-[3-(4-oxo-3,4-dihydro-phthalazin-1-ylamino)-propyl]-propionamide; 3-{3-[4-(2-morpholin-4-yl-ethoxy)-phenyl]-[1,2,4]oxadiazol-5-yl}-N-[3-(4-oxo-3,4-dihydro-phthalazin-1-ylamino)-propyl]-propionamide; 3-[3-(4-bromo-phenyl)-[1,2,4]oxadiazol-5-yl]-N-[3-(4-oxo-3,4-dihydro-phthalazin-1-ylamino)-propyl]-propionamide; 3-[3-(1,5-dimethyl-1H-pyrrol-2-yl)-[1,2,4]oxadiazol-5-yl]-N-[3-(4-oxo-3,4-dihydro-phthalazin-1-ylamino)-propyl]-propionamide; N-[2-hydroxy-3-(4-oxo-3,4-dihydro-phthalazin-1-ylamino)-propyl]-3-[3-(4-methylsulfanyl-phenyl)-[1,2,4]oxadiazol-5-yl]-propionamide; 3-[3-(4-fluorophenyl)-[1,2,4]oxadiazol-5-yl]-N-[2-hydroxy-3-(4-oxo-3,4-dihydro-phthalazin-1-ylamino)-propyl]-propionamide; 3-[3-(2,3-dihydro-benzofuran-5-yl)-[1,2,4]oxadiazol-5-yl]-N-[3-(4-oxo-3,4-dihydro-phthalazin-1-ylamino)-propyl]-propionamide; 1-[3-(5-oxo-5,6-dihydro-pyrido[2,3-d]pyridazin-8-ylamino)-propyl]-3-(2-oxo-tetrahydrofuran-3-yl)-thiourea; 3-[3-(6-dimethylamino-pyridin-3-yl)-[1,2,4]oxadiazol-5-yl]-N-[3-(4-oxo-3,4-dihydro-phthalazin-1-ylamino)-propyl]-propionamide; 3-[3-(4-acetylamino-phenyl)-[1,2,4]oxadiazol-5-yl]-N-[3-(4-oxo-3,4-dihydro-phthalazin-1-ylamino)-propyl]-propionamide; 5-oxo-5,6-dihydro-pyrido[2,3-d]pyridazin-8-ylamino)-cyclohexyl]-propionamide; [3-(5-oxo-5,6-dihydro-pyrido[2,3-d]pyridazin-8-ylamino)-propyl]-amide; 2-hydroxy-4-methylsulfanyl-N-[3-(5-oxo-5,6-dihydro-pyrido[2,3-d]pyridazin-8-ylamino)-propyl]-butyramide; 2-hydroxy-4-methylsulfanyl-N-[3-(8-oxo-7,8-dihydro-pyrazino[2,3-d]pyridazin-5-ylamino)-propyl]-butyramide; N-[3-(5-oxo-5,6-dihydro-pyrido[2,3-d]pyridazin-8-ylamino)-cyclohexyl]-propionamide; N-[2,2-dimethyl-3-(4-oxo-3,4-dihydro-phthalazin-1-ylamino)-propyl]-4-[3-(4-methoxy-phenyl)-[1,2,4]oxadiazol-5-yl]-butyramide; N-[3-(4-oxo-3,4-dihydro-phthalazin-1-ylamino)-propyl]-4-(3-pyridin-2-yl-[1,2,4]oxadiazol-5-yl)-butyramide; 4-[3-(3-nitro-phenyl)-[1,2,4]oxadiazol-5-yl]-N-[2-(4-oxo-3,4-dihydro-phthalazin-1-ylamino)-ethyl]-butyramide; 4-(3-{[3-(4-tert-butyl-phenyl)-[1,2,4]oxadiazol-5-ylmethyl]-amino}-propylamino)-2H-phthalazin-1-one; and 4-[3-(3,5-dichloro-phenyl)-[1,2,4]oxadiazol-5-yl]-N-[2-(4-oxo-3,4-dihydro-phthalazin-1-ylamino)-ethyl]-butyramide.

In yet another aspect, the PARP inhibitor comprises a benzimidazole derivative, as disclosed in U.S. Pat. No. 6,737,421, which is incorporated herein in its entirety by reference. Non-limiting examples of such a benzimidazole derivative include 2-(cis-4-amino-1-cyclohexyl)benzimidazole-4-carboxamide HCl; 2-(3-methoxycyclohexyl)benzimidazole-4-carboxamide; 2(4-methoxycyclohexyl)benzimidazole-4-carboxamide; 2-(4-(2-(N,N-diethylamino)ethoxy)cyclohexyl)benzimidazole-4-carboxamide 2HCl; trans-2-(4-aminocyclohexyl)benzimidazole-4-carboxamide; trans-2-(4-(aminomethyl)cyclohexyl)benzimidazole-4-carboxamide; 2-(4-methylcyclohexyl)benzimidazole-4-carboxamide; 2-(3-methylcyclohexyl)benzimidazole-4-carboxamide; 2-(2-methylcyclohexyl)benzimidazole-4-carboxamide; 2-(3-benzyloxyaminocyclohexyl)benzimidazole-4-carboxamide; 2-(3-aminocyclohexyl)benzimidazole-4-carboxamide HCl; 2-(cis-4-pyrrolidin-1-yl-1-cyclohexyl)benzimidazole-4-carboxamide; 2-(trans-4-pyrrolidin-1-yl-1-cyclohexyl)benzimidazole-4-carboxamide; 2-(cis-4-(piperazin-1-yl-1-cyclohexyl)benzimidazole-4-carboxamide; 2-(trans-4-(piperazin-1-yl-1-cylcyclohexyl)benzimidazole-4-carboxamide; 2-(4-(4-benzylpiperazin-1-yl-1-cyclohexyl)benzimidazole-4-carboxamide; 2-(3-(4-phenylpiperazin-1-yl-1-cyclohexyl)benzimidazole-4-carboxamide; 2-(3-(4-homopiperazin-1-yl-1-cyclohexyl)benzimidazole-4-carboxamide; 2-(4-(4-(N-methylhomopiperazin-1-yl)-1-cyclohexyl)benzimidazole-4-carboxamide; and 2-(3-(4-(4-phenyl-1,2,5,6-tetrahydropyridin-1-yl-1-cyclohexyl)benzimidazole-4-carboxamide.

In yet another aspect, the PARP inhibitor comprises a benzopyranophthalazinone derivative, as disclosed in U.S. Pat. No. 6,716,828, which is incorporated herein in its entirety by reference.

The concentration of a PARP inhibitor in such a pharmaceutical composition of the present invention can be in the range from about 0.001 to about 10 percent by weight of the total composition (or, alternatively, from about 0.001 to about 5 percent, or from about 0.01 to about 3 percent, or from about 0.01 to about 2 percent, or from about 0.1 to about 1 percent, or from about 0.01 to about 0.5 percent, by weight).

In one embodiment, a composition of the present invention is in a form of an emulsion, suspension, or dispersion. In another embodiment, the suspension or dispersion is based on an aqueous solution. For example, a composition of the present invention can comprise sterile saline solution.

In another aspect, a composition of the present invention can further comprise a non-ionic surfactant, such as polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myj® (, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S.C. Sweetman (Ed.), Pharmaceutical Press, London, 2005) and in Remington, “The Science and Practice of Pharmacy,” 21^{st} Ed., p. 291 and the contents of chapter 22, Lippincott Williams & Wilkins, New York, 2006); the contents of these sections are incorporated herein by reference. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1 weight percent).

In addition, a composition of the present invention can include additives such as buffers, diluents, carriers, adjuvants, or excipients. Any pharmacologically acceptable buffer suitable for application to the eye may be used. Other agents may be employed in the composition for a variety of purposes. For example, buffering agents, preservatives, co-solvents, oils, humectants, emollients, stabilizers, or antioxidants may be employed. Water-soluble preservatives that may be employed include sodium bisulfite, sodium bisulfate, sodium thiosulfate, benzalkonium chloride, chlorobutanol, thimerosal, ethyl alcohol, methylparaben, polyvinyl alcohol, benzyl alcohol, and phenylethyl alcohol. These agents may be present in individual amounts of from about 0.001 to about 5% by weight (preferably, from about 0.01% to about 2% by weight, or from about 0.01% to about 0.5% by weight). Suitable water-soluble buffering agents that may be employed are sodium carbonate, sodium borate, sodium phosphate, sodium acetate, sodium bicarbonate, etc., as approved by the United States Food and Drug Administration (“US FDA”) for the desired route of administration. These agents may be present in amounts sufficient to maintain a pH of the system of between about 2 and about 11. As such, the buffering agent may be as much as about 5% on a weight to weight basis of the total composition. Electrolytes such as, but not limited to, sodium chloride and potassium chloride may also be included in the formulation.

In one aspect, the pH of the composition is in the range from about 4.5 to about 11. Alternatively, the pH of the composition is in the range from about 6 to about 9, or from about 6.5 to about 8. In another aspect, the composition comprises a buffer having a pH in one of said pH ranges.

In another aspect, the composition has a pH of about 7. Alternatively, the composition has a pH in a range from about 7 to about 7.5.

In still another aspect, the composition has a pH of about 7.4.

In a further aspect, a composition of the present invention formulated for the treatment of dry eye-type diseases and disorders may also comprise carriers designed to provide immediate, short-term relief of dry eye-type conditions. Such carriers can be formulated as a phospholipid carrier or an artificial tears carrier, or mixtures of both. A phospholipid carrier comprises one or more phospholipids that lubricate, wet, approximate the consistency of endogenous tears, aid in natural tear build-up, or otherwise provide temporary relief of dry eye symptoms and conditions upon ocular administration. Non-limiting examples of phospholipid carrier formulations include those disclosed in U.S. Pat. Nos. 4,804,539; 4,883,658; 4,914,088; 5,075,104; 5,278,151; 5,294,607; 5,371,108; 5,578,586; the foregoing patents are incorporated herein by reference to the extent they disclose phospholipid compositions useful as phospholipid carriers of the present invention.

In yet another aspect, a composition also can comprise a viscosity-modifying compound designed to lubricate, wet, approximate the consistency of endogenous tears, aid in natural tear build-up, or otherwise provide temporary relief of dry eye symptoms and conditions upon ocular administration the eye. Such compounds may enhance the viscosity of the composition, and include, but are not limited to: monomeric polyols, such as, glycerol, propylene glycol, ethylene glycol; polymeric polyols, such as, polyethylene glycol; various polymers of the cellulose family, such as hydroxypropylmethyl cellulose (“HPMC”), carboxymethyl cellulose (“CMC”) sodium, hydroxypropyl cellulose (“HPC”); polysaccharides, such as hyaluronic acid and its salts, chondroitin sulfate and its salts, dextrans, such as, dextran 70; water soluble proteins, such as gelatin; vinyl polymers, such as, polyvinyl alcohol, polyvinylpyrrolidone, povidone; carbomers, such as carbomer 934P, carbomer 941, carbomer 940, or carbomer 974P; and acrylic acid polymers. In general, a desired viscosity can be in the range from about 1 to about 400 centipoises (“cp” or mPa·s).

In yet another aspect, the present invention provides a composition for treating, reducing, ameliorating, alleviating, or preventing the dry eye condition or an ophthalmic disorder requiring rewetting of the eye. The composition comprises: (a) at least a PARP inhibitor; and (b) a modulator of pro-inflammatory gene expression; said PARP inhibitor and said modulator of pro-inflammatory gene expression being present in amounts effective to treat, reduce, ameliorate, alleviate, or prevent said dry eye condition or ophthalmic disorder. In one embodiment, such a modulator of pro-inflammatory gene expression comprises an immunosuppressive medicament. In another embodiment, such an immunosuppressive medicament comprises Cyclosporine, such as for example Cyclosporine A. The concentration of Cyclosporine in such a composition can range from about 0.01 to about 2 percent by weight, or from about 0.1 to about 1.5 percent by weight, or from about 0.2 to about 1 percent by weight. Other immunosuppressive medicaments also can be suitable, such as Azathioprine, Cyclophosphamide, Tacrolimus Hydrate, Mycophenolate Mofetil, Mycophenolic Acid, Pimecrolimus (or its hydrate), or Sirolimus (or its hydrate). In one embodiment, an immunosuppressive medicament can be a biologically derived material, such as an immunoglobulin-containing antibody.

In still another embodiment, said modulator of pro-inflammatory gene expression comprises a dissociated glucocorticoid receptor agonist (“DIGRA”). Further details regarding DIGRAs are disclosed hereinbelow.

In still another aspect, a method for preparing a composition of the present invention comprises combining at least a PARP inhibitor with a pharmaceutically acceptable carrier. In one embodiment, such a carrier can be a sterile saline solution or a physiologically acceptable buffer. The step of combining can be carried out with equipment well known in the pharmaceutical art.

Physiologically acceptable buffers include, but are not limited to, a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5×PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO(N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In certain embodiments, a composition of the present invention is formulated in a buffer having a slight acidic pH, such as from about 6 to about 6.8. In such embodiments, the buffer capacity of the composition desirably allows the composition to come rapidly to a physiological pH after being administered to into the patient.

In yet another aspect, the present invention provides a composition for treating, reducing, ameliorating, alleviating, or preventing the dry eye condition or an ophthalmic disorder requiring rewetting of the eye. The composition comprises: (a) at least a PARP inhibitor; and (b) a dissociated glucocorticoid receptor agonist (“DIGRA”); said PARP inhibitor and DIGRA being present in amounts effective to treat, reduce, ameliorate, alleviate, or prevent said dry eye condition or ophthalmic disorder. The concentration of a DIGRA in such a composition can range from about 0.01 to about 2 percent by weight, or from about 0.1 to about 1.5 percent by weight, or from about 0.1 to about 1 percent by weight.

As used herein, a dissociated glucocorticoid receptor agonist (“DIGRA”) is a compound that is capable of binding to the glucocorticoid receptor (which is a polypeptide) and, upon binding, is capable of producing differentiated levels of transrepression and transactivation of gene expression. Thus, in one aspect, DIGRAs have the anti-inflammatory property similar to glucocorticosteroids but with lower levels of side effects of glucocorticosteroids. In another aspect, the DIGRA comprises a non-steroidal compound.

A useful DIGRA for a composition of the present invention can be any one of the compounds disclosed in U.S. Patent Application Publications 2004/0029932, 2004/0162321, 2004/0224992, 2005/0059714, 2005/0176706, 2005/0203128, 2005/0234091, 2005/0282881, 2006/0014787, 2006/0030561, 2006/0116396, 2006/0189646, 2006/0189647, and 2008/0009437, all of which are incorporated herein by reference in their entirety.

In certain embodiments, a DIGRA included in some compositions of the present invention has Formula XX or XXI.

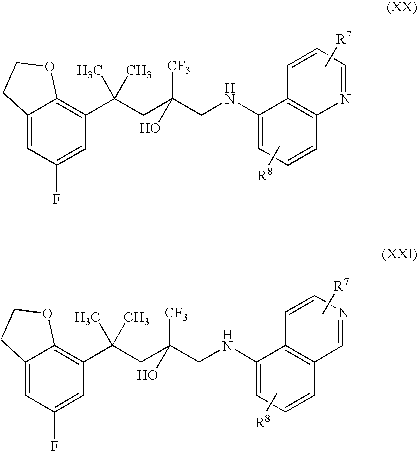

wherein R⁷ and R⁸ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C₅ or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups.

In still another embodiment, said DIGRA has Formula XXII.

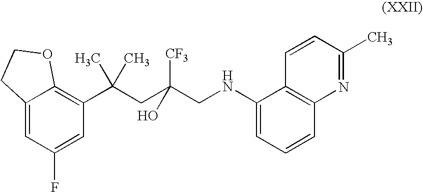

In another aspect, the present invention provides a method for preparing a composition that is suitable for the treatment, reduction, amelioration, alleviation, or prevention of a dry eye condition or a condition that requires the rewetting of the eye. The method comprises combining an inhibitor of PARP activity with a pharmaceutically acceptable carrier to form the composition. In certain embodiments, the method further comprises combining an additional material into the composition, wherein the additional material is selected from the group consisting of surfactants, buffers, diluents, adjuvants, expcipients, preservatives, co-solvents, oils, humectants, emollients, stabilizers, antioxidants, viscosity-modifying agents, and combinations thereof. The step of combining can be carried out in equipment commonly used in the production of pharmaceutical compositions, at conditions suitable for the particular ingredients.

The following examples provide some non-limiting compositions of the present invention.

### EXAMPLE 1

Two mixtures I and II are made separately by mixing the ingredients listed in Table 1. One part (by weight) of mixture I is mixed with twenty parts (by weight) of mixture II for 15 minutes or more. The pH of the combined mixture is adjusted to 6.2-6.4 using 1 N NaOH or 1 N HCl to yield a composition of the present invention.

TABLE 1

| Ingredient | Amount | |
|---|---|---|
| Mixture I | | |
| Carbopol 934P NF | 0.25 | g |
| Purified water | 99.75 | g |
| Mixture II | | |
| Propylene glycol | 5 | g |
| EDTA | 0.1 | mg |
| Compound of Formula I | 50 | g |

### EXAMPLE 2

Two mixtures I and II are made separately by mixing the ingredients listed in Table 2. One part (by weight) of mixture I is mixed with twenty parts (by weight) of mixture II for 15 minutes or more. The pH of the combined mixture is adjusted to 6.5-7 using 1 N NaOH or 1N HCl to yield a composition of the present invention.

TABLE 2

| Ingredient | Amount | |
|---|---|---|
| Mixture I | | |
| Carbopol 934P NF | 0.25 | g |
| Purified water | 99.75 | g |
| Mixture II | | |
| Propylene glycol | 5 | g |
| EDTA | 0.1 | mg |
| Compound of Formula IV | 50 | g |
| Cyclosporine A | 5 | g |

### EXAMPLE 3

Two mixtures I and II are made separately by mixing the ingredients listed in Table 3. One part (by weight) of mixture I is mixed with twenty parts (by weight) of mixture II for 15 minutes or more. The pH of the combined mixture is adjusted to 6.5-7.5 using 1 N NaOH or 1N HCl to yield a composition of the present invention.

TABLE 3

| Ingredient | Amount | |
|---|---|---|
| Mixture I | | |
| Carbopol 934P NF | 0.25 | g |
| Purified water | 99.75 | g |
| Mixture II | | |
| Propylene glycol | 3 | g |
| Triacetin | 7 | g |
| Compound of Formula XV | 50 | g |
| Cyclosporine A | 5 | g |
| EDTA | 0.1 | mg |

### EXAMPLE 4

Two mixtures I and II are made separately by mixing the ingredients listed in Table 4. Two parts (by weight) of mixture I are mixed with thirty parts (by weight) of mixture II for 15 minutes or more. The pH of the combined mixture is adjusted to 6.8-7.5 using 1 N NaOH or 1N HCl to yield a composition of the present invention.

TABLE 4

| Ingredient | Amount | |
|---|---|---|
| Mixture I | | |
| Carbopol 934P NF | 0.25 | g |
| Purified water | 99.75 | g |
| Mixture II | | |
| Propylene glycol | 7 | g |
| Glycerin | 3 | g |
| Compound of Formula XVIII | 50 | g |
| Compound of Formula XXII | 5 | g |
| HAP (30%) | 0.5 | mg |
| Alexidine 2HCl | 1-2 | ppm |

Note:
“HAP” denotes hydroxyalkyl phosphonates, such as those known under the trade name Dequest ®.

### EXAMPLE 5

The ingredients listed in Table 5 are mixed together for at least 15 minutes. The pH of the mixture is adjusted to 6.2-6.4 using 1 N NaOH or 1N HCl to yield a composition of the present invention.

TABLE 5

| Ingredient | Amount (% by weight) |
|---|---|
| Povidone | 1 |
| HAP (30%) | 0.05 |
| Glycerin | 3 |
| Propylene glycol | 3 |
| Compound of Formula XVII | 0.5 |
| Cyclosporine A | 0.1 |
| Compound of Formula XXII | 0.2 |
| Tyloxapol | 0.25 |
| BAK | 10-100 ppm |
| Purified water | q.s. to 100 |

Note:
“BAK” denotes benzalkonium chloride.

### EXAMPLE 6

The ingredients listed in Table 6 are mixed together for at least 15 minutes. The pH of the mixture is adjusted to 6.2-6.4 using 1 N NaOH or 1 N HCl to yield a composition of the present invention.

TABLE 6

| Ingredient | Amount (% by weight) |
|---|---|
| Povidone | 1.5 |
| HAP (30%) | 0.05 |
| Glycerin | 3 |
| Propylene glycol | 3 |
| 3-styryl-4-(3-piperidino-2-hydroxpropyl)- | 0.75 |
| Δ²-1,2,4-oxadiazolin-5-one | |
| Cyclosporine A | 0.1 |
| Tyloxapol | 0.25 |
| Alexidine 2HCl | 1-2 ppm |
| Purified water | q.s. to 100 |

### EXAMPLE 7

The ingredients listed in Table 7 are mixed together for at least 15 minutes. The pH of the mixture is adjusted to 6.2-6.4 using 1 N NaOH or 1 N HCl to yield a composition of the present invention.

TABLE 7

| Ingredient | Amount (% by weight) |
|---|---|
| CMC (MV) | 0.5 |
| HAP (30%) | 0.05 |
| Glycerin | 3 |
| Propylene glycol | 3 |
| 2-(4(4-n-propylpiperazin-1-yl)phenyl)-1H- | 0.75 |
| indole-4-carboxamide | |
| Compound of Formula XXII | 0.1 |
| Tyloxapol (a surfactant) | 0.25 |
| Alexidine 2HCl | 1-2 ppm |
| Purified water | q.s. to 100 |

### EXAMPLE 8

The ingredients listed in Table 8 are mixed together for at least 15 minutes. The pH of the mixture is adjusted to 6.2-6.4 using 1 N NaOH or 1 N HCl to yield a composition of the present invention.

TABLE 8

| Ingredient | Amount (% by weight) |
|---|---|
| CMC (MV) | 0.5 |
| HAP (30%) | 0.05 |
| Glycerin | 3 |
| Propylene glycol | 3 |
| 2-(4-(4-n-propyl-piperazin-1-yl)-phenyl)- | 0.75 |
| imidazo[1,2-a]pyridine-8-carboxamide | |
| Compound XVII | 0.25 |
| Compound of Formula XXII | 0.1 |
| Tyloxapol (a surfactant) | 0.25 |
| Alexidine 2HCl | 1-2 ppm |
| Purified water | q.s. to 100 |

### EXAMPLE 9

The ingredients listed in Table 9 are mixed together for at least 15 minutes. The pH of the mixture is adjusted to 6.2-6.4 using 1 N NaOH or 1 N HCl to yield a composition of the present invention.

TABLE 9

| Ingredient | Amount (% by weight) |
|---|---|
| CMC (MV) | 0.5 |
| HAP (30%) | 0.05 |
| Glycerin | 3 |
| Propylene glycol | 3 |
| 3,4,5,6-tetrahydro-1H-azepino[5,4,3- | 0.75 |
| cd]indol-6-one | |
| Compound XVIII | 0.25 |
| Cyclosporine A | 0.1 |
| Tyloxapol (a surfactant) | 0.25 |
| Alexidine 2HCl | 1-2 ppm |
| Purified water | q.s. to 100 |

### EXAMPLE 10

The ingredients listed in Table 10 are mixed together for at least 15 minutes. The pH of the mixture is adjusted to 6.2-6.4 using 1 N NaOH or 1 N HCl to yield a composition of the present invention.

TABLE 10

| Ingredient | Amount (% by weight) |
|---|---|
| CMC (MV) | 0.5 |
| HAP (30%) | 0.05 |
| Glycerin | 3 |
| Propylene glycol | 3 |
| N-[3-(4-oxo-3,4-dihydro-phthalazin-1- | 0.75 |
| ylamino)-propyl]-3-[3-(1H-pyrrol-2-yl)- | |
| [1,2,4]oxadiazol-5-yl]-propionamide | |
| 2-(cis-4-amino-1- | 0.25 |
| cyclohexyl)benzimidazole-4-carboxamide | |
| HCl | |
| Compound of Formula XXII | 0.1 |
| Cyclosporine A | 0.1 |
| Tyloxapol (a surfactant) | 0.25 |
| Alexidine 2HCl | 1-2 ppm |
| Purified water | q.s. to 100 |

In another aspect, a PARP inhibitor is incorporated into an ophthalmic device (such as an ophthalmic implantable device) that comprises a biodegradable material, and the device is implanted into a subject to provide a long-term (e.g., longer than about 1 week, or longer than about 1, 2, 3, 4, 5, or 6 months) treatment of the chronic inflammatory condition. Such a device may be implanted by a skilled physician in the subject's ocular or periocular tissue.

In still another aspect, a method for treating, reducing, ameliorating, or alleviating a dry eye condition or an ophthalmic disorder, which has an etiology in inflammation, comprises: (a) providing a composition comprising a PARP inhibitor; and (b) administering to a subject an amount of the composition at a frequency sufficient to treat, reduce, ameliorate, or alleviate the dry eye condition or the ophthalmic disorder in the subject.

In one embodiment, the PARP inhibitor is selected from among those disclosed above.

In another embodiment, the PARP inhibitor is selected from among compounds that are capable of inhibiting an activation of PARP or an ability of PARP to participate in a pro-inflammatory gene expression.

In another embodiment, the composition further comprises a material selected from the group consisting of immunosuppressive agents, DIGRAs, and combinations thereof. The concentration of an immunosuppressive agent or DIGRA is selected from among the ranges disclosed above.

In another aspect, a composition of the present invention is administered topically under an eyelid or on the ocular surface of the subject. In still another aspect, a composition of the present invention is injected into the conjunctival tissue of the subject.

In yet another aspect, a composition of the present invention is administered topically once daily, more than once per day, once every other day, or once a week.

### Testing for Potential Side Effects

PARP inhibitors are not expected to generate side effects that have been seen with glucocorticoid therapy. However, such effects may still be assessed by a test disclosed below. One of the most frequent undesirable actions of a glucocorticoid therapy is steroid diabetes. The reason for this is the stimulation of gluconeogenesis in the liver by the induction of the transcription of hepatic enzymes involved in gluconeogenesis and metabolism of free amino acids that are produced from the degradation of proteins (catabolic action of glucocorticoids). A key enzyme of the catabolic metabolism in the liver is the tyrosine aminotransferase (“TAT”). The activity of this enzyme can be determined photometrically from cell cultures of treated rat hepatoma cells. Thus, the gluconeogenesis by a glucocorticoid can be compared to that of a PARP inhibitor by measuring the activity of this enzyme. For example, in one procedure, the cells are treated for 24 hours with the test substance (a PARP inhibitor or glucocorticoid), and then the TAT activity is measured. The TAT activities for the selected PARP inhibitor and glucocorticoid are then compared. Other hepatic enzymes can be used in place of TAT, such as phosphoenolpyruvate carboxykinase, glucose-6-phosphatase, or fructose-2,6-biphosphatase. Alternatively, the levels of blood glucose in an animal model may be measured directly and compared for individual subjects that are treated with a glucocorticoid for a selected condition and those that are treated with a PARP inhibitor for the same condition.

Another undesirable result of glucocorticoid therapy is increased IOP in the subject. IOP of subjects treated with glucocorticoid and PARP inhibitor for a condition may be measured directly and compared.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition comprising an inhibitor of PARP activity in an amount effective for treating, reducing, ameliorating, alleviating, or preventing in a subject a dry eye condition or a condition that requires rewetting of the eye.

2. The composition of claim 1, wherein the inhibitor of PARP activity comprises at least a compound selected from the group consisting of compounds having Formulae I through XVIII.

3. The composition of claim 1, wherein the inhibitor of PARP activity comprises at least a compound having Formula XIX 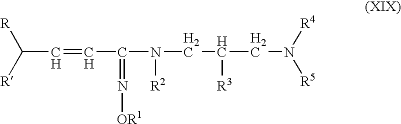
wherein R represents a C₁₋₂₀ alkyl group, a phenyl group, a phenyl group substituted by 1-3 substituents, wherein the substituent is selected from the group consisting of halogen atoms, C₁₋₃ alkyl groups, C₁₋₃ alkoxy groups, an amino group, (C₁₋₄ alkyl)amino groups, di(C₁₋₄ alkyl)amino groups, (C₁₋₄ alkanoyl)amino groups, and 5- or 6-membered saturated or unsaturated heterocyclic groups containing one or two nitrogen atoms or a sulphur atom as the heteroatom and each of said heterocyclic groups is optionally fused with one or more benzene rings, or one or more heterocyclic groups or one or more benzene rings and one or more heterocyclic groups; and R′ represents a hydrogen atom; or R forms together with R′ a C₅₋₇ cycloalkyl group optionally fused with a benzene ring; R⁴ and R⁵ represent, independently, a hydrogen atom, a C₁₋₅ alkyl group, a C₁₋₅ alkanoyl group, a phenyl group, a phenyl group substituted by 1-3 substituents, wherein the substituent is selected from the group consisting of halogen atoms, C₁₋₃ alkyl groups, and C₁₋₃ alkoxy groups; or R⁴ and R⁵ form together with the adjacent nitrogen atom a 5- or 6-membered saturated or unsaturated heterocyclic group that contains no or one additional heteroatom, wherein the additional heteroatom is selected from the group consisting of nitrogen, oxygen, and sulphur as the heteroatom, and that can be fused with a benzene ring, and each of the heterocyclic group and the benzene ring bears no, one, or two substituents, wherein the substituent is selected from the group consisting of halogen atoms, C₁₋₂ alkyl groups, C₁₋₂ alkoxy groups; and R¹, R², and R³ satisfy one of the following conditions: (i) R¹ and R² represent a hydrogen atom, R³ represents a hydrogen atom, a hydroxy group, or a C₁₋₅ alkoxy group; (ii) R¹ forms together with R² a carbonyl group or a thiocarbonyl group, the carbon atom of which is bound to the oxygen atom adjacent to R¹ and to the nitrogen atom adjacent to R², and R³ represents a hydrogen atom, a halogen atom, a hydroxy group, a C₁₋₅ alkoxy group, a C₁₋₅ alkylthio group, a C₁₋₂₀ alkanoyloxy group, a C₃₋₂₂ alkenoyloxy group containing one or more double bonds, a methylsulfonyloxy group, a benzenesulfonyloxy group, or a toluenesulfonyloxy group; or (iii) R² is a hydrogen atom and R¹ forms together with R³ a valence bond between the oxygen atom adjacent to R¹ and the carbon atom adjacent to R³.

4. The composition of claim 1, wherein the inhibitor of PARP activity comprises at least a compound selected from the group consisting of 3-styryl-4-(3-piperidino-2-hydroxpropyl)-Δ²-1,2,4-oxadiazolin-5-one; 3-styryl-4-(3-pyrrolidino-2-hydroxproxypropyl)-Δ²-1,2,4-oxadia-zolin-5-one; 3-styryl-4-(3-hexamethyleneimino-2-hydroxypropyl)-Δ²-1,2,4-oxa-diazolin-5-one; 3-styryl-4-(3-morpholino-2-hydroxypropyl)-Δ²-1,2,4-oxadiazolin-5-one; 3-styryl-4-[3-(tert.-butylamino)-2-hydroxypropyl]-Δ²-1,2,4-oxadiazolin-5-one; 3-styryl-4-{3-(1,2,3,4-tetrahydro-2-isoquinoyl)-2-hydroxypropyl-Δ²-1,2,4-oxadiazolin-5-one; 3-styryl-4-[3-(2,6-dimethylanilino)-2-hydroxypropyl]-Δ²-1,2,4-oxadiazolin-5-one; and 3-(3,4-dimethoxystyrl-4-(3-piperidino-2-hydroxypropyl)-Δ²-1,2,4-oxadiazolin-5-one.

5. The composition of claim 1, wherein the inhibitor of PARP activity comprises at least a compound selected from the group consisting of 2-(4(4-n-propylpiperazin-1-yl)phenyl)-1H-indole-4-carboxamide; 2-(4-piperazin-1-ylphenyl)-1H-indole-4-carboxamide; 2-(4(4-isopropylpiperazin-1-yl)phenyl)-1H-indole-4-carboxamide; 2-(4(4-benzylpiperazin-1-yl)phenyl)-1H-indole-4-carboxamide; 2-(4(4-n-butylpiperazin-1-yl)phenyl)-1H-indole-4-carboxamide; 2-(4(4-ethylpiperazin-1-yl)phenyl)-1H-indole-4-carboxamide; 2-(4-(2-N,N-dimethylaminoeth-1-yloxy)phenyl)-1H-indole-4-carboxamide; 2-(4-(2-pyrrolidin-1-yleth-1-yloxy)phenyl)-1H-indole-4-carboxamide; 2-(4-(2-piperidin-1-yleth-1-yloxy)phenyl)-1H-indole-4-carboxamide; 2-(4-(2-piperazin-1-yleth-1-yloxy)phenyl)-1H-indole-4-carboxamide; 2-(4-(2-(4-methylpiperazin-1-yl)eth-1-yloxy)phenyl)-1H-indole-4-carboxamide; 2-(4-(2-(4-propylpiperazin-1-yl)eth-1-yloxy)phenyl)-1H-indole-4-carboxamide; 2-(4-(2-(4-ethylpiperazin-1-yl)eth-1-yloxy)phenyl)-1H-indole-4-carboxamide; 2-(4-(2-(4-benzylpiperazin-1-yl)eth-1-yloxy)phenyl)-1H-indole-4-carboxamide; 2-(4-(2-(4-acetamidopiperazin-1-yl)eth-1-yloxy)phenyl)-1H-indole-4-carboxamide; 2-(4-(2-(4-benzanidopiperazin-1-yl)eth-1-yloxy)phenyl)-1H-indole-4-carboxamide; 2-(4-homopiperazin-1-ylphenyl)-1H-indole-4-carboxamide; 2-(4(4-methylhomopiperazin-1-yl)phenyl)-1H-indole-4-carboxamide; 2-(4(4-benzylhomopiperazin-1-yl)phenyl)-1H-indole-4-carboxamide; 2-(4-(4-n-butylhomopiperazin-1-yl)phenyl)-1H-indole-4-carboxamide; 2-(4(4-ethylhomopiperazin-1-yl)phenyl)-1H-indole-4-carboxamide; 2-(4-methoxyphenyl)-1H-indole-4-carboxamide; 2-(4-chlorophenyl)-1H-indole-4-carboxamide; 2-(4-aminophenyl)-1H-indole-4-carboxamide; 2-(4-methylphenyl)-1H-indole-4-carboxamide; 2-(4-phenylphenyl)-1H-indole-4-carboxamide; 2-(4-isopropylphenyl)-1H-indole-4-carboxamide; 2-(4-fluorophenyl)-1H-indole-4-carboxamide; 2-(4-trifluoromethylphenyl)-1H-indole-4-carboxamide; 2-(3-methoxyphenyl)-1H-indole-4-carboxamide; 2-(3-chlorophenyl)-1H-indole-4-carboxamide; 2-(3-aminophenyl)-1H-indole-4-carboxamide; 2-(3-methylphenyl)-1H-indole-4-carboxamide; 2-(3-phenylphenyl)-1H-indole-4-carboxamide; 2-(3-isopropylphenyl)-1H-indole-4-carboxamide; 2-(3-fluorophenyl)-1H-indole-4-carboxamide; 2-(3-trifluoromethylphenyl)-1H-indole-4-carboxamide; 2-piperidin-4-yl-1H-indole-4-carboxamide; 2-(1-methylpiperidin-4-yl)-1H-indole-4-carboxamide; 2-(1-n-propylpiperidin-4-yl)-1H-indole-4-carboxamide; 2-(1-benzylpiperidin-4-yl)-1H-indole-4-carboxamide; 2-(1-n-butylpiperidin-4-yl)-1H-indole-4-carboxamide; 2-(1-isopropylpiperidin-4-yl)-1H-indole-4-carboxamide; 2-pyridin-4-yl-1H-indole-4-carboxamide; 2-pyridin-3-yl-1H-indole-4-carboxamide; 2-pyridin-2-yl-1H-indole-4-carboxamide; 2-thien-2-yl-1H-indole-4-carboxamide; 2-thien-3-yl-1H-indole-4-carboxamide; 2-indol-3-yl-1H-indole-4-carboxamide; 2-indol-5-yl-1H-indole-4-carboxamide; 2-indol-2-yl-1H-indole-4-carboxamide; 2-quinolin-3-yl-1H-indole-4-carboxamide; 2-quinolin-2-yl-1H-indole-4-carboxamide; 2-quinolin-4-yl-1H-indole-4-carboxamide; 2-isoquinolin-1-yl-1H-indole-4-carboxamide; 2-isoquinolin-3-yl-1H-indole-4-carboxamide; 2-quinoxalin-2-yl-1H-indole-4-carboxamide; 2-naphth-2-yl-1H-indole-4-carboxamide; 2-naphth-1-yl-1H-indole-4-carboxamide; 2-(2(N,N-dimethylamino)eth-1-ylamino)phenyl)-1H-indole-4-carboxamide; 2-(2(N,N-diethylamino)eth-1-ylamino)phenyl)-1H-indole-4-carboxamide; 2-(2-piperidin-1-yleth-1-ylamino)phenyl)-1H-indole-4-carboxamide; 2-(2-pyrrolidin-1-yleth-1-ylamino)phenyl)-1H-indole-4-carboxamide; 2-(3(N,N-dimethylamino)prop-1-ylamino)phenyl)-1H-indole-4-carboxamide; 2-(3(N,N-diethylamino)prop-1-ylamino)phenyl)-1H-indole-4-carboxamide; 2-(3-piperidin-1-ylprop-1-ylamino)phenyl)-1H-indole-4-carboxamide; 2-(3-pyrrolidin-1-ylprop-1-ylamino)phenyl)-1H-indole-4-carboxamide; 2-cyclohexyl-1H-indole-4-carboxamide; 2-(cis-4-aminocyclohex-1-yl)-1H-indole-4-carboxamide; 2-(4-methoxycyclohex-1-yl)-1H-indole-4-carboxamide; 2-(4(4-n-propylpiperazin-1-yl)phenyl)-1,3,4,5-tetrahydro-6H-azepino[5,4,3-c,d]indol-6-one; 2-(4-piperazin-1-ylphenyl)-1,3,4,5-tetrahydro-6H-azepino[5,4,3-c,d]indol-6-one; 2-(4(4-isopropylpiperazin-1-yl)phenyl)-1,3,4,5-tetrahydro-6H-azepino[5,4,3-c,d]indol-6-one; 2-(4(4-benzylpiperazin-1-yl)phenyl)-1,3,4,5-tetrahydro-6H-azepino[5,4,3-c,d]indol-6-one; 2-(4(4-n-butylpiperazin-1-yl)phenyl)-1,3,4,5-tetrahydro-6H-azepino[5,4,3-c,d]indol-6-one; 2-(4(4-ethylpiperazin-1-yl)phenyl)-1,3,4,5-tetrahydro-6H-azepino[5,4,3-c,d]indol-6-one; 2-(4-(2-N,N-dimethylaminoeth-1-yloxy)phenyl)-1,3,4,5-tetrahydro-6H-azepino[5,4,3-c,d]indol-6-one; 2-(4-(2-pyrrolidin-1-yleth-1-yloxy)phenyl)-1,3,4,5-tetrahydro-6H-azepino[5,4,3-c,d]indol-6-one; 2-(4-(2-piperidin-1-yleth-1-yloxy)phenyl)-1,3,4,5-tetrahydro-6H-azepino[5,4,3-c,d]indol-6-one; 2-(4-(2-piperazin-1-yleth-1-yloxy)phenyl)-1,3,4,5-tetrahydro-6H-azepino[5,4,3-c,d]indol-6-one; 2-(4-(2-(4-methylpiperazin-1-yl)eth-1-yloxy)phenyl)-1,3,4,5-tetrahydro-6H-azepino[5,4,3-c,d]indol-6-one; 2-(4-(2-(4-propylpiperazin-1-yl)eth-1-yloxy)phenyl)-1,3,4,5-tetrahydro-6H-azepino[5,4,3-c,d]indol-6-one; 2-(4-(2-(4-ethylpiperazin-1-yl)eth-1-yloxy)phenyl)-1,3,4,5-tetrahydro-6H-azepino[5,4,3-c,d]indol-6-one; 2-(4-(2-(4-benzylpiperazin-1-yl)eth-1-yloxy)phenyl)-1,3,4,5-tetrahydro-6H-azepino[5,4,3-c,d]indol-6-one; 2-(4-(2-(4-acetamidopiperazin-1-yl)eth-1-yloxy)phenyl)-1,3,4,5-tetrahydro-6H-azepino[5,4,3-c,d]indol-6-one; 2-(4-(2-(4-benzamidopiperazin-2-yl)eth-1-yloxy)phenyl)-1,3,4,5-tetrahydro-6H-azepino[5,4,3-c,d]indol-6-one; 2-(4-homopiperazin-1-ylphenyl)-1,3,4,5-tetrahydro-6H-azepino[5,4,3-c,d]indol-6-one; 2-(4(4-Mmthylhomopiperazin-1-yl)phenyl)-1,3,4,5-tetrahydro-6H-azepino[5,4,3-c,d]indol-6-one; 2-(4(4-benzylhomopiperazin-1-yl)phenyl)-1,3,4,5-tetrahydro-6H-azepino[5,4,3-c,d]indol-6-one; 2-(4-(4-n-butylhomopiperazin-1-yl)phenyl)-1,3,4,5-tetrahydro-6H-azepino[5,4,3-c,d]indol-6-one; 2-(4(4-ethylhomopiperazin-1-yl)phenyl)-1,3,4,5-tetrahydro-6H-azepino[5,4,3-c,d]indol-6-one; 2-piperidin-4-yl-1,3,4,5-tetrahydro-6H-azepino[5,4,3-c,d]indol-6-one; 2-(1-methylpiperidin-4-yl)-1,3,4,5-tetrahydro-6H-azepino[5,4,3-c,d]indol-6-one; 2-(1-n-propylpiperidin-4-yl)-1,3,4,5-tetrahydro-6H-azepino[5,4,3-c,d]indol-6-one; 2-(1-benzylpiperidin-4-yl)-1,3,4,5-tetrahydro-6H-azepino[5,4,3-c,d]indol-6-one; 2-(1-n-butylpiperidin-4-yl)-1,3,4,5-tetrahydro-6H-azepino[5,4,3-c,d]indol-6-one; 2-(1-isopropylpiperidin-4-yl)-1,3,4,5-tetrahydro-6H-azepino[5,4,3-c,d]indol-6-one; 2-pyridin-4-yl-1,3,4,5-tetrahydro-6H-azepino[5,4,3-c,d]indol-6-one; 2-pyridin-3-yl-1,3,4,5-tetrahydro-6H-azepino[5,4,3-c,d]indol-6-one; 2-pyridin-2-yl-1,3,4,5-tetrahydro-6H-azepino[5,4,3-c,d]indol-6-one; 2-thien-2-yl-1,3,4,5-tetrahydro-6H-azepino[5,4,3-c,d]indol-6-one; 2-thien-3-yl-1,3,4,5-tetrahydro-6H-azepino[5,4,3-c,d]indol-6-one; 2-indol-3-yl-1,3,4,5-tetrahydro-6H-azepino[5,4,3-c,d]indol-6-one; 2-indol-5-yl-1,3,4,5-tetrahydro-6H-azepino[5,4,3-c,d]indol-6-one; 2-indol-2-yl-1,3,4,5-tetrahydro-6H-azepino[5,4,3-c,d]indol-6-one; 2-quinolin-3-yl-1,3,4,5-tetrahydro-6H-azepino[5,4,3-c,d]indol-6-one; 2-quinolin-2-yl-1,3,4,5-tetrahydro-6H-azepino[5,4,3-c,d]indol-6-one; 2-quinolin-4-yl-1,3,4,5-tetrahydro-6H-azepino[5,4,3-c,d]indol-6-one; 2-isoquinolin-1-yl-1,3,4,5-tetrahydro-6H-azepino[5,4,3-c,d]indol-6-one; 2-isoquinolin-3-yl-1,3,4,5-tetrahydro-6H-azepino[5,4,3-c,d]indol-6-one; 2-quinoxalin-2-yl-1,3,4,5-tetrahydro-6H-azepino[5,4,3-c,d]indol-6-one; 2-naphth-2-yl-1,3,4,5-tetrahydro-6H-azepino[5,4,3-c,d]indol-6-one; 2-naphth-1-yl-1,3,4,5-tetrahydro-6H-azepino[5,4,3-c,d]indol-6-one; 2-(2(N,N-dimethylamino)eth-1-ylamino)phenyl)-1,3,4,5-tetrahydro-6H-azepino[5,4,3-c,d]indol-6-one; 2-(2(N,N-diethylamino)eth-1-ylamino)phenyl)-1,3,4,5-tetrahydro-6H-azepino[5,4,3-c,d]indol-6-one; 2-(2-piperidin-1-yleth-1-ylamino)phenyl)-1,3,4,5-tetrahydro-6H-azepino[5,4,3-c,d]indol-6-one; 2-(2-pyrrolidin-1-yleth-1-ylamino)phenyl)-1,3,4,5-tetrahydro-6H-azepino[5,4,3-c,d]indol-6-one; 2-(3 (N,N-dimethylamino)prop-1-ylamino)phenyl)-1,3,4,5-tetrahydro-6H-azepino[5,4,3-c,d]indol-6-one; 2-(3 (N,N-diethylamino)prop-1-ylamino)phenyl)-1,3,4,5-tetrahydro-6H-azepino[5,4,3-c,d]indol-6-one; 2-(3-piperidin-1-ylprop-1-ylamino)phenyl)-1,3,4,5-tetrahydro-6H-azepino[5,4,3-c,d]indol-6-one; 2-(3-pyrrolidin-1-ylprop-1-ylamino)phenyl)-1,3,4,5-tetrahydro-6H-azepino[5,4,3-c,d]indol-6-one; 2-cyclohexyl-1,3,4,5-tetrahydro-6H-azepino[5,4,3-c,d]indol-6-one; 2-(cis-4-aminocyclohex-1-yl)-1,3,4,5-tetrahydro-6H-azepino[5,4,3-c,d]indo-1-6-one; and 2-(4-methoxycyclohex-1-yl)-1,3,4,5-tetrahydro-6H-azepino[5,4,3-c,d]indol-6-one.

6. The composition of claim 1, wherein the inhibitor of PARP activity comprises at least a compound selected from the group consisting of 2-(4-(4-n-propyl-piperazin-1-yl)-phenyl)-imidazo[1,2-a]pyridine-8-carboxamide; 2-(4-piperazin-1-yl-phenyl)-imidazo[1,2-a]pyridine-8-carboxamide; 2-(4-(4-isopropyl-piperazin-1-yl)-phenyl)-imidazo[1,2-a]pyridine-8-carboxamide; 2-(4-(4-benzylpiperazin-1-yl)-phenyl)-imidazo[1,2-a]pyridine-8-carboxamide; 2-(4-(4-n-butyl-piperazin-1-yl)-phenyl)-imidazo[1,2-a]pyridine-8-carboxamide; 2-(4-(4-ethyl-piperazin-1-yl)-phenyl)-imidazo[1,2-a]pyridine-8-carboxamide; 2-(4-(2-N,N-dimethylaminoeth-1-yloxy)-phenyl)-imidazo-[1,2-a]pyridine-8-carboxamide; 2-(4-(2-pyrrolidin-1-yl-eth-1-yloxy)-phenyl)-imidazo[1,2-a]pyridine-8-carboxamide; 2-(4-(2-piperidin-1-yl-eth-1-yloxy)-phenyl)-imidazo[1,2-a]pyridine-8-carboxamide; 2-(4-(2-piperazin-1-yl-eth-1-yloxy)-phenyl)-imidazo[1,2-a]pyridine-8-carboxamide; 2-(4-(2-(4-methyl-piperazin-1-yl)-eth-1-yloxy)-phenyl)-imidazo[1,2-a]pyridine-8-carboxamide; 2-(4-(2-(4-propyl-piperazin-1-yl)-eth-1-yloxy)-phenyl-imidazo[1,2-a]pyridine-8-carboxamide; 2-(4-(2-(4-ethyl-piperazin-1-yl)-eth-1-yloxy)-phenyl-imidazo[1,2-a]pyridine-8-carboxamide; 2-(4-(2-(4-benzyl-piperazin-1-yl)-eth-1-yloxy)-phenyl-imidazo[1,2-a]pyridine-8-carboxamide; 2-(4-(2-(4-acetamido-piperazin-1-yl)-eth-1-yloxy)-phenyl-imidazo[1,2-a]pyridine-8-carboxamide; 2-(4-(2-(4-benzamido-piperazin-1-yl)-eth-1-yloxy)-phenyl-imidazo[1,2-a]pyridine-8-carboxamide; 2-(4-homopiperazin-1-yl-phenyl)-imidazo[1,2-a]pyridine-8-carboxamide; 2-(4(4-methylhomopiperazin-1-yl)-phenyl)-imidazo[1,2-a]pyridine-8-carboxamide; 2-(4(4-benzylhomopiperazin-1-yl)-phenyl)-imidazo[1,2-a]pyridine-8-carboxamide; 2-(4-(4-n-butyl-homopiperazin-1-yl)-phenyl)-imidazo[1,2-a]pyridine-8-carboxamide; 2-(4(4-ethylhomo-piperazin-1-yl)-phenyl)-imidazo[1,2-a]pyridine-8-carboxamide; 2-(4-methoxy-phenyl)-imidazo[1,2-a]pyridine-8-carboxamide; 2-(4-methyl-phenyl)-imidazo[1,2-a]pyridine-8-carboxamide; 2-(4-phenyl-phenyl)-imidazo[1,2-a]pyridine-8-carboxamide; 2-(4-isopropyl-phenyl)-imidazo[1,2-a]pyridine-8-carboxamide; 2-(4-fluoro-phenyl)-imidazo[1,2-a]pyridine-8-carboxamide; 2-(4-trifluoromethyl-phenyl)-imidazo[1,2-a]pyridine-8-carboxamide; 2-(3-methoxy-phenyl)-imidazo[1,2-a]pyridine-8-carboxamide; 2-(3-chloro-phenyl)-imidazo[1,2-a]pyridine-8-carboxamide; 2-(3-amino-phenyl)-imidazo[1,2-a]pyridine-8-carboxamide; 2-(3-methyl-phenyl)-imidazo[1,2-a]pyridine-8-carboxamide; 2-(3-phenyl-phenyl)-imidazo[1,2-a]pyridine-8-carboxamide; 2-(3-isopropyl-phenyl)-imidazo[1,2-a]pyridine-8-carboxamide; 2-(3-fluoro-phenyl)-imidazo[1,2-a]pyridine-8-carboxamide; 2-(3-trifluoromethyl-phenyl)-imidazo[1,2-a]pyridine-8-carboxamide; 2-piperidin-4-yl-imidazo[1,2-a]pyridine-8-carboxamide; 2-(1-methyl-piperidin-4-yl)-imidazo[1,2-a]pyridine-8-carboxamide; 2-(1-ethyl-piperidin-4-yl)-imidazo[1,2-a]pyridine-8-carboxamide; 2-(1-n-propyl-piperidin-4-yl)-imidazo[1,2-a]pyridine-8-carboxamide; 2-(1-benzyl-piperidin-4-yl)-imidazo[1,2-a]pyridine-8-carboxamide; 2-(1-n-butyl-piperidin-4-yl)-imidazo[1,2-a]pyridine-8-carboxamide; 2-(1-isopropyl-piperidin-4-yl)-imidazo[1,2-a]pyridine-8-carboxamide; 2-pyridin-4-yl-imidazo[1,2-a]pyridine-8-carboxamide; 2-pyridin-3-yl-imidazo[1,2-a]pyridine-8-carboxamide; 2-pyridin-2-yl-imidazo[1,2-a]pyridine-8-carboxamide; 2-thien-2-yl-imidazo[1,2-a]pyridine-8-carboxamide; 2-thien-3-yl-imidazo[1,2-a]pyridine-8-carboxamide; 2-indol-3-yl-imidazo[1,2-a]pyridine-8-carboxamide; 2-indol-5-yl-imidazo[1,2-a]pyridine-8-carboxamide; 2-indol-2-yl-imidazo[1,2-a]pyridine-8-carboxamide; 2-quinolin-3-yl-imidazo[1,2-a]pyridine-8-carboxamide; 2-quinolin-2-yl-imidazo[1,2-a]pyridine-8-carboxamide; 2-quinolin-4-yl-imidazo[1,2-a]pyridine-8-carboxamide; 2-isoquinolin-1-yl-imidazo[1,2-a]pyridine-8-carboxamide; 2-isoquinolin-3-yl-imidazo[1,2-a]pyridine-8-carboxamide; 2-quinoxalin-2-yl-imidazo[1,2-a]pyridine-8-carboxamide; 2-naphth-2-yl-imidazo[1,2-a]pyridine-8-carboxamide; 2-naphth-1-yl-imidazo[1,2-a]pyridine-8-carboxamide; 2-(4-(2(N,N-dimethylamino)-eth-1-ylamino)-phenyl)-imidazo[1,2-a]pyridine-8-carboxamide; 2-(4-(2(N,N-diethylamino)-eth-1-ylamino)-phenyl)-imidazo[1,2-a]pyridine-8-carboxamide; 2-(4-(2-piperidin-1-yl-eth-1-ylamino)-phenyl)-imidazo[1,2-a]pyridine-8-carboxamide; 2-(4-(2-pyrrolidin-1-yl-eth-1-ylamino)-phenyl)-imidazo[1,2-a]pyridine-8-carboxamide; 2-(4-(3(N,N-dimethylamino)-prop-1-ylamino)-phenyl)-imidazo[1,2-a]pyridine-8-carboxamide; 2-(4-(3 (N,N-diethylamino)-prop-1-ylamino)-phenyl)-imidazo[1,2-a]pyridine-8-carboxamide; 2-(4-(3-piperidin-1-yl-prop-1-ylamino)-phenyl)-imidazo[1,2-a]pyridine-8-carboxamide; 2-(4-(3-pyrrolidin-1-yl-prop-1-ylamino)-phenyl)-imidazo-[1,2-a]pyridine-8-carboxamide; 2-cyclohexyl-imidazo[1,2-a]pyridine-8-carboxamide; 2-(cis-4-amino-cyclohex-1-yl)-imidazo[1,2-a]pyridine-8-carboxamide; 2-(4-methoxy-cyclohex-1-yl)-imidazo[1,2-a]pyridine-8-carboxamide; 2-(4-(2(N,N-dimethylamino)-eth-1-yl-methylamino)-phenyl)-imidazo[1,2-a]-pyridine-8-carboxamide; and 2-(4-(4-methyl-piperazin-1-yl)-phenyl)-imidazo[1,2-a]pyridine-8-carboxamide.

7. The composition of claim 1, wherein the inhibitor of PARP activity comprises at least a compound selected from the group consisting of 3,4-dihydropyrrolo[4,3,2-de]isoquinolin-5-(1H)-one; 2-bromo-3,4-dihydropyrrolo[4,3,2-de]isoquinolin-5-(1H)-one; phenyl-3,4-dihydropyrrolo[4,3,2-de]isoquinolin-5-(1H)-one; 3,4,5,6-tetrahydro-1H-azepino[5,4,3-cd]indol-6-one; 2-bromo-3,4,5,6-tetrahydro-1H-azepino[5,4,3-cd]indol-6-one; 2-phenyl-3,4,5,6-tetrahydro-1H-azepino[5,4,3-cd]indol-6-one; 2-(4-Methoxyphenyl)-3,4,5,6-tetrahydro-1H-azepino[5,4,3-cd]indol-6-one; 2-(3-nitrophenyl)-3,4,5,6-tetrahydro-1H-azepino[5,4,3-cd]indol-6-one; 2-(3-hydroxymethylphenyl)-3,4,5,6-tetrahydro-1H-azepino[5,4,3-cd]indol-6-one; 2-(phenylethynyl)-3,4,5,6-tetrahydro-1H-azepino[5,4,3-cd]indol-6-one; 1-methyl-2-phenyl-3,4,5,6-tetrahydro-1H-azepino[5,4,3-cd]indol-6-one; 1-N-methyl-3,4,5,6-tetrahydro-1H-azepino[5,4,3-cd]indol-6-one; (rac)-3-phenyl-3,4,5,6-tetrahydro-1H-azepino[5,4,3-cd]indol-6-one; 2-(4-fluorophenyl)-3,4,5,6-tetrahydro-1H-azepino[5,4,3-cd]indol-6-one; 8-bromo-2-phenyl-3,4,5,6-tetrahydro-1H-azepino[5,4,3-cd]indol-6-one; 2-(4-(N,N-dimethylamino)methylphenyl)-3,4,5,6-tetrahydro-1H-azepino[5,4,3-c d]indol-6-one; 2-(3-(N,N-dimethylamino)methylphenyl)-3,4,5,6-tetrahydro-1H-azepino[5,4,3-cd]indol-6-one; 1,5-dihydro-[1,2]diazepino[4,5,6-cd]-indol-6-one; 1,5-dihydro-3-phenyl-[1,2]diazepino[4,5,6-cd]-indol-6-one; 1,5-dihydro-3-phenethyl-[1,2]diazepino[4,5,6-cd]-indol-6-one; 2-(3-trifluoromethyl-phenyl)-1,3,4,5-tetrahydro-azepino[5,4,3-cd]indol-6-one; 2-(4-trifluoromethyl-phenyl)-1,3,4,5-tetrahydro-azepino[5,4,3-cd]indol-6-one; 2-benzofuran-2-yl-1,3,4,5-tetrahydro-azepino[5,4,3-cd]indol-6-one; 2-(3,5-bis-trifluoromethyl-phenyl)-1,3,4,5-tetrahydro-azepino[5,4,3-cd]indo 1-6-one; 2-(4-bromophenyl)-1,3,4,5-tetrahydro-azepino[5,4,3-cd]indol-6-one; 2-(3-chloro-4-fluoro-phenyl)-1,3,4,5-tetrahydro-azepino[5,4,3-cd]indol-6-one; 2-(4-tert-butyl-phenyl)-1,3,4,5-tetrahydro-azepino[5,4,3-cd]indol-6-one; 2-phenyl-1,3,4,5-tetrahydro-azepino[5,4,3-cd]indole-6-thione; 2-phenethyl-1,3,4,5-tetrahydro-azepino[5,4,3-cd]indol-6-one; 2-(2-chlorophenyl)-1,3,4,5-tetrahydro-azepino[5,4,3-cd]indol-6-one; 2-naphthalen-1-yl-1,3,4,5-tetrahydro-azepino[5,4,3-cd]indol-6-one; 6-oxo-3,4,5,6-tetrahydro-1H-azepino[5,4,3-cd]indole-2-carboxylic acid methyl ester; 2-iodo-1,3,4,5-tetrahydro-azepino[5,4,3-cd]indol-6-one; 2-(4-(N-methylamino)methylphenyl)-3,4,5,6-tetrahydro-1H-azepino[5,4,3-cd]indol-6-one; 2-(3-(N-methylamino)methylphenyl)-3,4,5,6-tetrahydro-1H-azepino[5,4,3-cd]indol-6-one; 2-(3-(3-piperidin-1-ylmethylphenyl)-3,4,5,6-tetrahydro-1H-azepino[5,4,3-cd]indol-6-one; N-[3-(6-oxo-3,4,5,6-tetrahydro-1H-azepino[5,4,3-cd]indol-2-yl)-phenyl]-acetamide; 3-[2-(6-oxo-3,4,5,6-tetrahydro-1H-azepino[5,4,3-cd]indol-2-yl)-phenyl]-propionic acid methyl ester; 2-pyridin-3-yl-1,3,4,5-tetrahydro-azepino[5,4,3-cd]indol-6-one; 2-(2-methylsulfanyl-phenyl)-1,3,4,5-tetrahydro-azepino[5,4,3-cd]indol-6-one; 2-[4-(2-pyrrolidin-1-yl-ethyl)-phenyl]-1,3,4,5-tetrahydro-azepino[5,4,3-cd]indol-6-one; N-[4-fluoro-2-(6-oxo-3,4,5,6-tetrahydro-1H-azepino[5,4,3-cd]indol-2-yl)-phenyl]-acetamide; 6-oxo-3,4,5,6-tetrahydro-1H-azepino[5,4,3-cd]indole-2-carboxylic acid methylamide; 4-(6-oxo-3,4,5,6-tetrahydro-1H-azepino[5,4,3-cd]indol-2-yl)-benzoic acid; 6-oxo-3,4,5,6-tetrahydro-1H-azepino[5,4,3-cd]indole-2-carboxylic acid; 6-oxo-3,4,5,6-tetrahydro-1H-azepino[5,4,3-cd]indole-2-carboxylic acid (4-fluoro-phenyl)-amide; 2-(1H-pyrrol-2-yl)-1,3,4,5-tetrahydro-azepino[5,4,3-cd]indol-6-one; (RS)-(.+−.)-1-(4-chloro-phenyl)-9-(4-methoxy-phenyl)-8,9-dihydro-7H-2,7,9a-triaza-benzo[cd]azulen-6-one; (3-[1,3]dioxolan-2-yl-phenyl)-8,9-dihydro-7H-2,7,9a-triaza-benzo[cd]azulen-6-one; 1-(4-diethoxymethyl-phenyl)-8,9-dihydro-7H-2,7,9a-triaza-benzo[cd]azulen-6-one; 1-(3-pyrrolidin-1-ylmethyl-phenyl)-8,9-dihydro-7H-2,7,9a-triaza-benzo[cd]azulen-6-one; 1-[3-(3-trifluoromethyl-phenoxy)-phenyl]-8,9-dihydro-7H-2,7,9a-triaza-benzo[cd]azulen-6-one; 1-(3-[1,3]dioxolan-2-yl-phenyl)-4-fluoro-8,9-dihydro-7H-2,7,9a-triaza-benzo[cd]azulen-6-one; 1-(3-dimethylaminomethyl-phenyl)-4-fluoro-8,9-dihydro-7H-2,7,9a-triaza-benzo[cd]azulen-6-one; 1-(4-morpholin-4-ylmethyl-phenyl)-8,9-dihydro-7H-2,7,9a-triaza-benzo[cd]azulen-6-one; 1-(3-p-tolyl-benzo[c]isoxazol-5-yl)-8,9-dihydro-7H-2,7,9a-triaza-benzo[cd]azulen-6-one; 4-[5-(6-oxo-6,7,8,9-tetrahydro-2,7,9a-triaza-benzo[cd]azulen-1-yl)-pyridin-2-yloxy]-benzonitrile; 6-oxo-6,7,8,9-tetrahydro-2,7,9a-triaza-benzo[cd]azulen-1-carboxylic acid benzylamide; 1-(4-methyl-piperazine-1-carbonyl)-8,9-dihydro-7H-2,7,9a-triaza-benzo[cd]azulen-6-one; 1-[4-(2-pyrrolidin-1-yl-ethyl)-phenyl]-8,9-dihydro-7H-2,7,9a-triaza-benzo[c d]azulen-6-one; 1-[4-((2S,5S)-2,5-bis-methoxymethyl-pyrrolidin-1-ylmethyl)-phenyl]-8,9-dihydro-7H-2,7,9a-triaza-benzo[cd]azulen-6-one; and (R)-1-(4-dimethylaminomethyl-phenyl)-8-hydroxymethyl-8,9-dihydro-7H-2,7,9a-triaza-benzo[cd]azulen-6-one.

8. The composition of claim 1, wherein the inhibitor of PARP activity comprises at least a compound selected from the group consisting of N-[3-(4-oxo-3,4-dihydro-phthalazin-1-ylamino)-propyl]-3-[3-(1H-pyrrol-2-yl)-[1,2,4]oxadiazol-5-yl]-propionamide; N-[3-(4-oxo-3,4-dihydro-phthalazin-1-ylamino)-propyl]-3-(3-thiophen-3-yl-[1,2,4]oxadiazol-5-yl)-propionamide; 3-[3-(2-methyl-thiophen-3-yl)-[1,2,4]oxadiazol-5-yl]-N-[3-(4-oxo-3,4-dihydro-phthalazin-1-ylamino)-propyl]-propionamide; 3-[3-(4-methanesulfonylamino-phenyl)-[1,2,4]oxadiazol-5-yl]-N-[3-(4-oxo-3,4-dihydro-phthalazin-1-ylamino)-propyl]-propionamide; 3-{3-[4-(2-morpholin-4-yl-ethoxy)-phenyl]-[1,2,4]oxadiazol-5-yl}-N-[3-(4-oxo-3,4-dihydro-phthalazin-1-ylamino)-propyl]-propionamide; 3-[3-(4-bromo-phenyl)-[1,2,4]oxadiazol-5-yl]-N-[3-(4-oxo-3,4-dihydro-phthalazin-1-ylamino)-propyl]-propionamide; 3-[3-(1,5-dimethyl-1H-pyrrol-2-yl)-[1,2,4]oxadiazol-5-yl]-N-[3-(4-oxo-3,4-dihydro-phthalazin-1-ylamino)-propyl]-propionamide; N-[2-hydroxy-3-(4-oxo-3,4-dihydro-phthalazin-1-ylamino)-propyl]-3-[3-(4-methylsulfanyl-phenyl)-[1,2,4]oxadiazol-5-yl]-propionamide; 3-[3-(4-fluorophenyl)-[1,2,4]oxadiazol-5-yl]-N-[2-hydroxy-3-(4-oxo-3,4-dihydro-phthalazin-1-ylamino)-propyl]-propionamide; 3-[3-(2,3-dihydro-benzofuran-5-yl)-[1,2,4]oxadiazol-5-yl]-N-[3-(4-oxo-3,4-dihydro-phthalazin-1-ylamino)-propyl]-propionamide; 1-[3-(5-oxo-5,6-dihydro-pyrido[2,3-d]pyridazin-8-ylamino)-propyl]-3-(2-oxo-tetrahydrofuran-3-yl)-thiourea; 3-[3-(6-dimethylamino-pyridin-3-yl)-[1,2,4]oxadiazol-5-yl]-N-[3-(4-oxo-3,4-dihydro-phthalazin-1-ylamino)-propyl]-propionamide; 3-[3-(4-acetylamino-phenyl)-[1,2,4]oxadiazol-5-yl]-N-[3-(4-oxo-3,4-dihydro-phthalazin-1-ylamino)-propyl]-propionamide; 5-oxo-5,6-dihydro-pyrido[2,3-d]pyridazin-8-ylamino)-cyclohexyl]-propionamide; [3-(5-oxo-5,6-dihydro-pyrido[2,3-d]pyridazin-8-ylamino)-propyl]-amide; 2-hydroxy-4-methylsulfanyl-N-[3-(5-oxo-5,6-dihydro-pyrido[2,3-d]pyridazin-8-ylamino)-propyl]-butyramide; 2-hydroxy-4-methylsulfanyl-N-[3-(8-oxo-7,8-dihydro-pyrazino[2,3-d]pyridazin-5-ylamino)-propyl]-butyramide; N-[3-(5-oxo-5,6-dihydro-pyrido[2,3-d]pyridazin-8-ylamino)-cyclohexyl]-propionamide; N-[2,2-dimethyl-3-(4-oxo-3,4-dihydro-phthalazin-1-ylamino)-propyl]-4-[3-(4-methoxy-phenyl)-[1,2,4]oxadiazol-5-yl]-butyramide; N-[3-(4-oxo-3,4-dihydro-phthalazin-1-ylamino)-propyl]-4-(3-pyridin-2-yl-[1,2,4]oxadiazol-5-yl)-butyramide; 4-[3-(3-nitro-phenyl)-[1,2,4]oxadiazol-5-yl]-N-[2-(4-oxo-3,4-dihydro-phthalazin-1-ylamino)-ethyl]-butyramide; 4-(3-{[3-(4-tert-butyl-phenyl)-[1,2,4]oxadiazol-5-ylmethyl]-amino}propylamino)-2H-phthalazin-1-one; and 4-[3-(3,5-dichloro-phenyl)-[1,2,4]oxadiazol-5-yl]-N-[2-(4-oxo-3,4-dihydro-phthalazin-1-ylamino)-ethyl]-butyramide.

9. The composition of claim 1, wherein the inhibitor of PARP activity comprises at least a compound selected from the group consisting of 2-(cis-4-amino-1-cyclohexyl)benzimidazole-4-carboxamide HCl; 2-(3-methoxycyclohexyl)benzimidazole-4-carboxamide; 2(4-methoxycyclohexyl)benzimidazole-4-carboxamide; 2-(4-(2-(N,N-diethylamino)ethoxy)cyclohexyl)benzimidazole-4-carboxamide 2HCl; trans-2-(4-aminocyclohexyl)benzimidazole-4-carboxamide; trans-2-(4-(aminomethyl)cyclohexyl)benzimidazole-4-carboxamide; 2-(4-methylcyclohexyl)benzimidazole-4-carboxamide; 2-(3-methylcyclohexyl)benzimidazole-4-carboxamide; 2-(2-methylcyclohexyl)benzimidazole-4-carboxamide; 2-(3-benzyloxyaminocyclohexyl)benzimidazole-4-carboxamide; 2-(3-aminocyclohexyl)benzimidazole-4-carboxamide HCl; 2-(cis-4-pyrrolidin-1-yl-1-cyclohexyl)benzimidazole-4-carboxamide; 2-(trans-4-pyrrolidin-1-yl-1-cyclohexyl)benzimidazole-4-carboxamide; 2-(cis-4-(piperazin-1-yl-1-cyclohexyl)benzimidazole-4-carboxamide; 2-(trans-4-(piperazin-1-yl-1-cylcyclohexyl)benzimidazole-4-carboxamide; 2-(4-(4-benzylpiperazin-1-yl-1-cyclohexyl)benzimidazole-4-carboxamide; 2-(3-(4-phenylpiperazin-1-yl-1-cyclohexyl)benzimidazole-4-carboxamide; 2-(3-(4-homopiperazin-1-yl-1-cyclohexyl)benzimidazole-4-carboxamide; 2-(4-(4-(N-methylhomopiperazin-1-yl)-1-cyclohexyl)benzimidazole-4-carboxamide; and 2-(3-(4-(4-phenyl-1,2,5,6-tetrahydropyridin-1-yl-1-cyclohexyl)benzimidazole-4-carboxamide.

10. The composition of claim 1, further comprises a modulator of a pro-inflammatory gene expression.

11. The composition of claim 10, wherein the modulator of a pro-inflammatory gene expression comprises an immunosuppressive medicament.

12. The composition of claim 11, wherein the immunosuppressive medicament comprises a material selected from the group consisting of Cyclosporine, Azathioprine, Cyclophosphamide, Tacrolimus Hydrate, Mycophenolate Mofetil, Mycophenolic Acid, Pimecrolimus, Sirolimus, Pimecrolimus Hydrate, Sirolimus Hydrate, immunoglobulin antibodies, combinations thereof, and mixtures thereof.

13. The composition of claim 11, wherein the immunosuppressive medicament comprises Cyclosporine A.

14. The composition of claim 10, wherein the modulator of a pro-inflammatory gene expression comprises a dissociated glucocorticoid receptor agonist (“DIGRA”).

15. The composition of claim 14, wherein the DIGRA comprises a compound having Formula XX or XXI 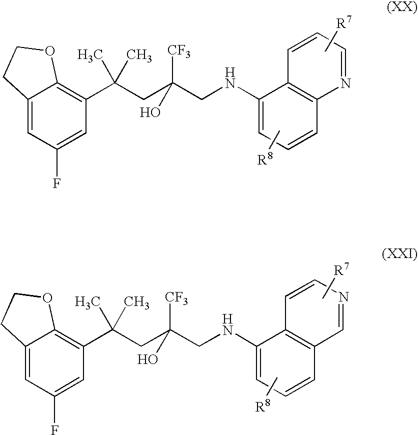
wherein R⁷ and R⁸ are independently selected from the group consisting of hydrogen, halogen, cyano, hydroxy, C₁-C₁₀ alkoxy groups, unsubstituted C₁-C₁₀ linear or branched alkyl groups, substituted C₁-C₁₀ linear or branched alkyl groups, unsubstituted C₃-C₁₀ cyclic alkyl groups, and substituted C₃-C₁₀ cyclic alkyl groups.

16. The composition of claim 14, wherein the DIGRA comprises a compound having Formula XXII

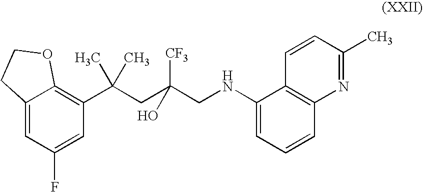

17. The composition of claim 1, wherein said inhibitor of PARP activity is present in an amount in a range from about 0.001 to about 10 percent by weight of the total composition.

18. The composition of claim 1, wherein said inhibitor of PARP activity is present in an amount in a range from about 0.01 to about 2 percent by weight of the total composition.

19. The composition of claim 17, wherein the composition comprises a solution, dispersion, emulsion, suspension, or implant.

20. A method for treating, reducing, ameliorating, alleviating, or preventing a dry eye condition or an ophthalmic disorder, which has an etiology in inflammation, the method comprising: (a) providing a composition comprising a an inhibitor of PARP activity; and (b) administering to a subject an amount of the composition at a frequency sufficient to treat, reduce, ameliorate, alleviate, or prevent the dry eye condition or the ophthalmic disorder in the subject.

21. The method of claim 20, wherein the inhibitor of PARP activity comprises at least a compound selected from the group consisting of compounds having Formulae I through XVIII.

22. The method of claim 20, wherein the inhibitor of PARP activity comprises at least a compound having Formula XIX 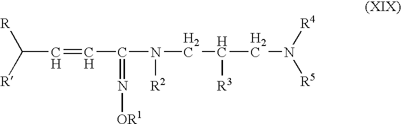
wherein R represents a C₁₋₂₀ alkyl group, a phenyl group, a phenyl group substituted by 1-3 substituents, wherein the substituent is selected from the group consisting of halogen atoms, C₁₋₃ alkyl groups, C₁₋₃ alkoxy groups, an amino group, (C₁₋₄ alkyl)amino groups, di(C₁₋₄ alkyl)amino groups, (C₁₋₄ alkanoyl)amino groups, and 5- or 6-membered saturated or unsaturated heterocyclic groups containing one or two nitrogen atoms or a sulphur atom as the heteroatom and each of said heterocyclic groups is optionally fused with one or more benzene rings, or one or more heterocyclic groups or one or more benzene rings and one or more heterocyclic groups; and R′ represents a hydrogen atom; or R forms together with R′ a C₅₋₇ cycloalkyl group optionally fused with a benzene ring; R⁴ and R⁵ represent, independently, a hydrogen atom, a C₁₋₅ alkyl group, a C₁₋₅ alkanoyl group, a phenyl group, a phenyl group substituted by 1-3 substituents, wherein the substituent is selected from the group consisting of halogen atoms, C₁₋₃ alkyl groups, and C₁₋₃ alkoxy groups; or R⁴ and R⁵ form together with the adjacent nitrogen atom a 5- or 6-membered saturated or unsaturated heterocyclic group that contains no or one additional heteroatom, wherein the additional heteroatom is selected from the group consisting of nitrogen, oxygen, and sulphur as the heteroatom, and that can be fused with a benzene ring, and each of the heterocyclic group and the benzene ring bears no, one, or two substituents, wherein the substituent is selected from the group consisting of halogen atoms, C₁₋₂ alkyl groups, C₁₋₂ alkoxy groups; and R¹, R², and R³ satisfy one of the following conditions: (i) R¹ and R² represent a hydrogen atom, R³ represents a hydrogen atom, a hydroxy group, or a C₁₋₅ alkoxy group; (ii) R¹ forms together with R² a carbonyl group or a thiocarbonyl group, the carbon atom of which is bound to the oxygen atom adjacent to R¹ and to the nitrogen atom adjacent to R², and R³ represents a hydrogen atom, a halogen atom, a hydroxy group, a C₁₋₅ alkoxy group, a C₁₋₅ alkylthio group, a C₁₋₂₀ alkanoyloxy group, a C₃₋₂₂ alkenoyloxy group containing one or more double bonds, a methylsulfonyloxy group, a benzenesulfonyloxy group, or a toluenesulfonyloxy group; or (iii) R² is a hydrogen atom and R′ forms together with R³ a valence bond between the oxygen atom adjacent to R¹ and the carbon atom adjacent to R³.

23. The method of claim 22, wherein the inhibitor of PARP activity comprises at least a compound selected from the group consisting of 3-styryl-4-(3-piperidino-2-hydroxpropyl)-Δ²-1,2,4-oxadiazolin-5-one; 3-styryl-4-(3-pyrrolidino-2-hydroxproxypropyl)-Δ²-1,2,4-oxadia-zolin-5-one; 3-styryl-4-(3-hexamethyleneimino-2-hydroxypropyl)-Δ²-1,2,4-oxa-diazolin-5-one; 3-styryl-4-(3-morpholino-2-hydroxypropyl)-Δ²-1,2,4-oxadiazolin-5-one; 3-styryl-4-[3-(tert.-butylamino)-2-hydroxypropyl]-Δ²-1,2,4-oxadiazolin-5-one; 3-styryl-4-{3-(1,2,3,4-tetrahydro-2-isoquinoyl)-2-hydroxypropyl-Δ²-1,2,4-oxadiazolin-5-one; 3-styryl-4-[3-(2,6-dimethylanilino)-2-hydroxypropyl]-Δ²-1,2,4-oxadiazolin-5-one; and 3-(3,4-dimethoxystyrl-4-(3-piperidino-2-hydroxypropyl)-Δ²-1,2,4-oxadiazolin-5-one.

24. The method of claim 20, wherein the inhibitor of PARP activity comprises at least a compound selected from the group consisting of 2-(4(4-n-propylpiperazin-1-yl)phenyl)-1H-indole-4-carboxamide; 2-(4-piperazin-1-ylphenyl)-1H-indole-4-carboxamide; 2-(4(4-isopropylpiperazin-1-yl)phenyl)-1H-indole-4-carboxamide; 2-(4(4-benzylpiperazin-1-yl)phenyl)-1H-indole-4-carboxamide; 2-(4(4-n-butylpiperazin-1-yl)phenyl)-1H-indole-4-carboxamide; 2-(4(4-ethylpiperazin-1-yl)phenyl)-1H-indole-4-carboxamide; 2-(4-(2-N,N-dimethylaminoeth-1-yloxy)phenyl)-1H-indole-4-carboxamide; 2-(4-(2-pyrrolidin-1-yleth-1-yloxy)phenyl)-1H-indole-4-carboxamide; 2-(4-(2-piperidin-1-yleth-1-yloxy)phenyl)-1H-indole-4-carboxamide; 2-(4-(2-piperazin-1-yleth-1-yloxy)phenyl)-1H-indole-4-carboxamide; 2-(4-(2-(4-methylpiperazin-1-yl)eth-1-yloxy)phenyl)-1H-indole-4-carboxamide; 2-(4-(2-(4-propylpiperazin-1-yl)eth-1-yloxy)phenyl)-1H-indole-4-carboxamide; 2-(4-(2-(4-ethylpiperazin-1-yl)eth-1-yloxy)phenyl)-1H-indole-4-carboxamide; 2-(4-(2-(4-benzylpiperazin-1-yl)eth-1-yloxy)phenyl)-1H-indole-4-carboxamide; 2-(4-(2-(4-acetamidopiperazin-1-yl)eth-1-yloxy)phenyl)-1H-indole-4-carboxamide; 2-(4-(2-(4-benzanidopiperazin-1-yl)eth-1-yloxy)phenyl)-1H-indole-4-carboxamide; 2-(4-homopiperazin-1-ylphenyl)-1H-indole-4-carboxamide; 2-(4(4-methylhomopiperazin-1-yl)phenyl)-1H-indole-4-carboxamide; 2-(4(4-benzylhomopiperazin-1-yl)phenyl)-1H-indole-4-carboxamide; 2-(4-(4-n-butylhomopiperazin-1-yl)phenyl)-1H-indole-4-carboxamide; 2-(4(4-ethylhomopiperazin-1-yl)phenyl)-1H-indole-4-carboxamide; 2-(4-methoxyphenyl)-1H-indole-4-carboxamide; 2-(4-chlorophenyl)-1H-indole-4-carboxamide; 2-(4-aminophenyl)-1H-indole-4-carboxamide; 2-(4-methylphenyl)-1H-indole-4-carboxamide; 2-(4-phenylphenyl)-1H-indole-4-carboxamide; 2-(4-isopropylphenyl)-1H-indole-4-carboxamide; 2-(4-fluorophenyl)-1H-indole-4-carboxamide; 2-(4-trifluoromethylphenyl)-1H-indole-4-carboxamide; 2-(3-methoxyphenyl)-1H-indole-4-carboxamide; 2-(3-chlorophenyl)-1H-indole-4-carboxamide; 2-(3-aminophenyl)-1H-indole-4-carboxamide; 2-(3-methylphenyl)-1H-indole-4-carboxamide; 2-(3-phenylphenyl)-1H-indole-4-carboxamide; 2-(3-isopropylphenyl)-1H-indole-4-carboxamide; 2-(3-fluorophenyl)-1H-indole-4-carboxamide; 2-(3-trifluoromethylphenyl)-1H-indole-4-carboxamide; 2-piperidin-4-yl-1H-indole-4-carboxamide; 2-(1-methylpiperidin-4-yl)-1H-indole-4-carboxamide; 2-(1-n-propylpiperidin-4-yl)-1H-indole-4-carboxamide; 2-(1-benzylpiperidin-4-yl)-1H-indole-4-carboxamide; 2-(1-n-butylpiperidin-4-yl)-1H-indole-4-carboxamide; 2-(1-isopropylpiperidin-4-yl)-1H-indole-4-carboxamide; 2-pyridin-4-yl-1H-indole-4-carboxamide; 2-pyridin-3-yl-1H-indole-4-carboxamide; 2-pyridin-2-yl-1H-indole-4-carboxamide; 2-thien-2-yl-1H-indole-4-carboxamide; 2-thien-3-yl-1H-indole-4-carboxamide; 2-indol-3-yl-1H-indole-4-carboxamide; 2-indol-5-yl-1H-indole-4-carboxamide; 2-indol-2-yl-1H-indole-4-carboxamide; 2-quinolin-3-yl-1H-indole-4-carboxamide; 2-quinolin-2-yl-1H-indole-4-carboxamide; 2-quinolin-4-yl-1H-indole-4-carboxamide; 2-isoquinolin-1-yl-1H-indole-4-carboxamide; 2-isoquinolin-3-yl-1H-indole-4-carboxamide; 2-quinoxalin-2-yl-1H-indole-4-carboxamide; 2-naphth-2-yl-1H-indole-4-carboxamide; 2-naphth-1-yl-1H-indole-4-carboxamide; 2-(2(N,N-dimethylamino)eth-1-ylamino)phenyl)-1H-indole-4-carboxamide; 2-(2(N,N-diethylamino)eth-1-ylamino)phenyl)-1H-indole-4-carboxamide; 2-(2-piperidin-1-yleth-1-ylamino)phenyl)-1H-indole-4-carboxamide; 2-(2-pyrrolidin-1-yleth-1-ylamino)phenyl)-1H-indole-4-carboxamide; 2-(3 (N,N-dimethylamino)prop-1-ylamino)phenyl)-1H-indole-4-carboxamide; 2-(3(N,N-diethylamino)prop-1-ylamino)phenyl)-1H-indole-4-carboxamide; 2-(3-piperidin-1-ylprop-1-ylamino)phenyl)-1H-indole-4-carboxamide; 2-(3-pyrrolidin-1-ylprop-1-ylamino)phenyl)-1H-indole-4-carboxamide; 2-cyclohexyl-1H-indole-4-carboxamide; 2-(cis-4-aminocyclohex-1-yl)-1H-indole-4-carboxamide; 2-(4-methoxycyclohex-1-yl)-1H-indole-4-carboxamide; 2-(4(4-n-propylpiperazin-1-yl)phenyl)-1,3,4,5-tetrahydro-6H-azepino[5,4,3-c,d]indol-6-one; 2-(4-piperazin-1-ylphenyl)-1,3,4,5-tetrahydro-6H-azepino[5,4,3-c,d]indol-6-one; 2-(4(4-isopropylpiperazin-1-yl)phenyl)-1,3,4,5-tetrahydro-6H-azepino[5,4,3-c,d]indol-6-one; 2-(4(4-benzylpiperazin-1-yl)phenyl)-1,3,4,5-tetrahydro-6H-azepino[5,4,3-c,d]indol-6-one; 2-(4(4-n-butylpiperazin-1-yl)phenyl)-1,3,4,5-tetrahydro-6H-azepino[5,4,3-c,d]indol-6-one; 2-(4(4-ethylpiperazin-1-yl)phenyl)-1,3,4,5-tetrahydro-6H-azepino[5,4,3-c,d]indol-6-one; 2-(4-(2-N,N-dimethylaminoeth-1-yloxy)phenyl)-1,3,4,5-tetrahydro-6H-azepino[5,4,3-c,d]indol-6-one; 2-(4-(2-pyrrolidin-1-yleth-1-yloxy)phenyl)-1,3,4,5-tetrahydro-6H-azepino[5,4,3-c,d]indol-6-one; 2-(4-(2-piperidin-1-yleth-1-yloxy)phenyl)-1,3,4,5-tetrahydro-6H-azepino[5,4,3-c,d]indol-6-one; 2-(4-(2-piperazin-1-yleth-1-yloxy)phenyl)-1,3,4,5-tetrahydro-6H-azepino[5,4,3-c,d]indol-6-one; 2-(4-(2-(4-methylpiperazin-1-yl)eth-1-yl oxy)phenyl)-1,3,4,5-tetrahydro-6H-azepino[5,4,3-c,d]indol-6-one; 2-(4-(2-(4-propylpiperazin-1-yl)eth-1-yloxy)phenyl)-1,3,4,5-tetrahydro-6H-azepino[5,4,3-c,d]indol-6-one; 2-(4-(2-(4-ethylpiperazin-1-yl)eth-1-yloxy)phenyl)-1,3,4,5-tetrahydro-6H-azepino[5,4,3-c,d]indol-6-one; 2-(4-(2-(4-benzylpiperazin-1-yl)eth-1-yloxy)phenyl)-1,3,4,5-tetrahydro-6H-azepino[5,4,3-c,d]indol-6-one; 2-(4-(2-(4-acetamidopiperazin-1-yl)eth-1-yloxy)phenyl)-1,3,4,5-tetrahydro-6H-azepino[5,4,3-c,d]indol-6-one; 2-(4-(2-(4-benzamidopiperazin-2-yl)eth-1-yloxy)phenyl)-1,3,4,5-tetrahydro-6H-azepino[5,4,3-c,d]indol-6-one; 2-(4-homopiperazin-1-ylphenyl)-1,3,4,5-tetrahydro-6H-azepino[5,4,3-c,d]indol-6-one; 2-(4(4-Mmthylhomopiperazin-1-yl)phenyl)-1,3,4,5-tetrahydro-6H-azepino[5,4,3-c,d]indol-6-one; 2-(4(4-benzylhomopiperazin-1-yl)phenyl)-1,3,4,5-tetrahydro-6H-azepino[5,4,3-c,d]indol-6-one; 2-(4-(4-n-butylhomopiperazin-1-yl)phenyl)-1,3,4,5-tetrahydro-6H-azepino[5,4,3-c,d]indol-6-one; 2-(4(4-ethylhomopiperazin-1-yl)phenyl)-1,3,4,5-tetrahydro-6H-azepino[5,4,3-c,d]indol-6-one; 2-piperidin-4-yl-1,3,4,5-tetrahydro-6H-azepino[5,4,3-c,d]indol-6-one; 2-(1-methylpiperidin-4-yl)-1,3,4,5-tetrahydro-6H-azepino[5,4,3-c,d]indol-6-one; 2-(1-n-propylpiperidin-4-yl)-1,3,4,5-tetrahydro-6H-azepino[5,4,3-c,d]indol-6-one; 2-(1-benzylpiperidin-4-yl)-1,3,4,5-tetrahydro-6H-azepino[5,4,3-c,d]indol-6-one; 2-(1-n-butylpiperidin-4-yl)-1,3,4,5-tetrahydro-6H-azepino[5,4,3-c,d]indol-6-one; 2-(1-isopropylpiperidin-4-yl)-1,3,4,5-tetrahydro-6H-azepino[5,4,3-c,d]indol-6-one; 2-pyridin-4-yl-1,3,4,5-tetrahydro-6H-azepino[5,4,3-c,d]indol-6-one; 2-pyridin-3-yl-1,3,4,5-tetrahydro-6H-azepino[5,4,3-c,d]indol-6-one; 2-pyridin-2-yl-1,3,4,5-tetrahydro-6H-azepino[5,4,3-c,d]indol-6-one; 2-thien-2-yl-1,3,4,5-tetrahydro-6H-azepino[5,4,3-c,d]indol-6-one; 2-thien-3-yl-1,3,4,5-tetrahydro-6H-azepino[5,4,3-c,d]indol-6-one; 2-indol-3-yl-1,3,4,5-tetrahydro-6H-azepino[5,4,3-c,d]indol-6-one; 2-indol-5-yl-1,3,4,5-tetrahydro-6H-azepino[5,4,3-c,d]indol-6-one; 2-indol-2-yl-1,3,4,5-tetrahydro-6H-azepino[5,4,3-c,d]indol-6-one; 2-quinolin-3-yl-1,3,4,5-tetrahydro-6H-azepino[5,4,3-c,d]indol-6-one; 2-quinolin-2-yl-1,3,4,5-tetrahydro-6H-azepino[5,4,3-c,d]indol-6-one; 2-quinolin-4-yl-1,3,4,5-tetrahydro-6H-azepino[5,4,3-c,d]indol-6-one; 2-isoquinolin-1-yl-1,3,4,5-tetrahydro-6H-azepino[5,4,3-c,d]indol-6-one; 2-isoquinolin-3-yl-1,3,4,5-tetrahydro-6H-azepino[5,4,3-c,d]indol-6-one; 2-quinoxalin-2-yl-1,3,4,5-tetrahydro-6H-azepino[5,4,3-c,d]indol-6-one; 2-naphth-2-yl-1,3,4,5-tetrahydro-6H-azepino[5,4,3-c,d]indol-6-one; 2-naphth-1-yl-1,3,4,5-tetrahydro-6H-azepino[5,4,3-c,d]indol-6-one; 2-(2(N,N-dimethylamino)eth-1-ylamino)phenyl)-1,3,4,5-tetrahydro-6H-azepino[5,4,3-c,d]indol-6-one; 2-(2(N,N-diethylamino)eth-1-ylamino)phenyl)-1,3,4,5-tetrahydro-6H-azepino[5,4,3-c,d]indol-6-one; 2-(2-piperidin-1-yleth-1-ylamino)phenyl)-1,3,4,5-tetrahydro-6H-azepino[5,4,3-c,d]indol-6-one; 2-(2-pyrrolidin-1-yleth-1-ylamino)phenyl)-1,3,4,5-tetrahydro-6H-azepino[5,4,3-c,d]indol-6-one; 2-(3 (N,N-dimethylamino)prop-1-ylamino)phenyl)-1,3,4,5-tetrahydro-6H-azepino[5,4,3-c,d]indol-6-one; 2-(3(N,N-diethylamino)prop-1-ylamino)phenyl)-1,3,4,5-tetrahydro-6H-azepino[5,4,3-c,d]indol-6-one; 2-(3-piperidin-1-ylprop-1-ylamino)phenyl)-1,3,4,5-tetrahydro-6H-azepino[5,4,3-c,d]indol-6-one; 2-(3-pyrrolidin-1-ylprop-1-ylamino)phenyl)-1,3,4,5-tetrahydro-6H-azepino[5,4,3-c,d]indol-6-one; 2-cyclohexyl-1,3,4,5-tetrahydro-6H-azepino[5,4,3-c,d]indol-6-one; 2-(cis-4-aminocyclohex-1-yl)-1,3,4,5-tetrahydro-6H-azepino[5,4,3-c,d]indo-1-6-one; and 2-(4-methoxycyclohex-1-yl)-1,3,4,5-tetrahydro-6H-azepino[5,4,3-c,d]indol-6-one.

25. The method of claim 20, wherein the inhibitor of PARP activity comprises at least a compound selected from the group consisting of 2-(4-(4-n-propyl-piperazin-1-yl)-phenyl)-imidazo[1,2-a]pyridine-8-carboxamide; 2-(4-piperazin-1-yl-phenyl)-imidazo[1,2-a]pyridine-8-carboxamide; 2-(4-(4-isopropyl-piperazin-1-yl)-phenyl)-imidazo[1,2-a]pyridine-8-carboxamide; 2-(4-(4-benzylpiperazin-1-yl)-phenyl)-imidazo[1,2-a]pyridine-8-carboxamide; 2-(4-(4-n-butyl-piperazin-1-yl)-phenyl)-imidazo[1,2-a]pyridine-8-carboxamide; 2-(4-(4-ethyl-piperazin-1-yl)-phenyl)-imidazo[1,2-a]pyridine-8-carboxamide; 2-(4-(2-N,N-dimethylaminoeth-1-yloxy)-phenyl)-imidazo-[1,2-a]pyridine-8-carboxamide; 2-(4-(2-pyrrolidin-1-yl-eth-1-yloxy)-phenyl)-imidazo[1,2-a]pyridine-8-carboxamide; 2-(4-(2-piperidin-1-yl-eth-1-yloxy)-phenyl)-imidazo[1,2-a]pyridine-8-carboxamide; 2-(4-(2-piperazin-1-yl-eth-1-yloxy)-phenyl)-imidazo[1,2-a]pyridine-8-carboxamide; 2-(4-(2-(4-methyl-piperazin-1-yl)-eth-1-yloxy)-phenyl)-imidazo[1,2-a]pyridine-8-carboxamide; 2-(4-(2-(4-propyl-piperazin-1-yl)-eth-1-yloxy)-phenyl-imidazo[1,2-a]pyridine-8-carboxamide; 2-(4-(2-(4-ethyl-piperazin-1-yl)-eth-1-yloxy)-phenyl-imidazo[1,2-a]pyridine-8-carboxamide; 2-(4-(2-(4-benzyl-piperazin-1-yl)-eth-1-yloxy)-phenyl-imidazo[1,2-a]pyridine-8-carboxamide; 2-(4-(2-(4-acetamido-piperazin-1-yl)-eth-1-yloxy)-phenyl-imidazo[1,2-a]pyridine-8-carboxamide; 2-(4-(2-(4-benzamido-piperazin-1-yl)-eth-1-yloxy)-phenyl-imidazo[1,2-a]pyridine-8-carboxamide; 2-(4-homopiperazin-1-yl-phenyl)-imidazo[1,2-a]pyridine-8-carboxamide; 2-(4(4-methylhomopiperazin-1-yl)-phenyl)-imidazo[1,2-a]pyridine-8-carboxamide; 2-(4(4-benzylhomopiperazin-1-yl)-phenyl)-imidazo[1,2-a]pyridine-8-carboxamide; 2-(4-(4-n-butyl-homopiperazin-1-yl)-phenyl)-imidazo[1,2-a]pyridine-8-carboxamide; 2-(4(4-ethylhomo-piperazin-1-yl)-phenyl)-imidazo[1,2-a]pyridine-8-carboxamide; 2-(4-methoxy-phenyl)-imidazo[1,2-a]pyridine-8-carboxamide; 2-(4-methyl-phenyl)-imidazo[1,2-a]pyridine-8-carboxamide; 2-(4-phenyl-phenyl)-imidazo[1,2-a]pyridine-8-carboxamide; 2-(4-isopropyl-phenyl)-imidazo[1,2-a]pyridine-8-carboxamide; 2-(4-fluoro-phenyl)-imidazo[1,2-a]pyridine-8-carboxamide; 2-(4-trifluoromethyl-phenyl)-imidazo[1,2-a]pyridine-8-carboxamide; 2-(3-methoxy-phenyl)-imidazo[1,2-a]pyridine-8-carboxamide; 2-(3-chloro-phenyl)-imidazo[1,2-a]pyridine-8-carboxamide; 2-(3-amino-phenyl)-imidazo[1,2-a]pyridine-8-carboxamide; 2-(3-methyl-phenyl)-imidazo[1,2-a]pyridine-8-carboxamide; 2-(3-phenyl-phenyl)-imidazo[1,2-a]pyridine-8-carboxamide; 2-(3-isopropyl-phenyl)-imidazo[1,2-a]pyridine-8-carboxamide; 2-(3-fluoro-phenyl)-imidazo[1,2-a]pyridine-8-carboxamide; 2-(3-trifluoromethyl-phenyl)-imidazo[1,2-a]pyridine-8-carboxamide; 2-piperidin-4-yl-imidazo[1,2-a]pyridine-8-carboxamide; 2-(1-methyl-piperidin-4-yl)-imidazo[1,2-a]pyridine-8-carboxamide; 2-(1-ethyl-piperidin-4-yl)-imidazo[1,2-a]pyridine-8-carboxamide; 2-(1-n-propyl-piperidin-4-yl)-imidazo[1,2-a]pyridine-8-carboxamide; 2-(1-benzyl-piperidin-4-yl)-imidazo[1,2-a]pyridine-8-carboxamide; 2-(1-n-butyl-piperidin-4-yl)-imidazo[1,2-a]pyridine-8-carboxamide; 2-(1-isopropyl-piperidin-4-yl)-imidazo[1,2-a]pyridine-8-carboxamide; 2-pyridin-4-yl-imidazo[1,2-a]pyridine-8-carboxamide; 2-pyridin-3-yl-imidazo[1,2-a]pyridine-8-carboxamide; 2-pyridin-2-yl-imidazo[1,2-a]pyridine-8-carboxamide; 2-thien-2-yl-imidazo[1,2-a]pyridine-8-carboxamide; 2-thien-3-yl-imidazo[1,2-a]pyridine-8-carboxamide; 2-indol-3-yl-imidazo[1,2-a]pyridine-8-carboxamide; 2-indol-5-yl-imidazo[1,2-a]pyridine-8-carboxamide; 2-indol-2-yl-imidazo[1,2-a]pyridine-8-carboxamide; 2-quinolin-3-yl-imidazo[1,2-a]pyridine-8-carboxamide; 2-quinolin-2-yl-imidazo[1,2-a]pyridine-8-carboxamide; 2-quinolin-4-yl-imidazo[1,2-a]pyridine-8-carboxamide; 2-isoquinolin-1-yl-imidazo[1,2-a]pyridine-8-carboxamide; 2-isoquinolin-3-yl-imidazo[1,2-a]pyridine-8-carboxamide; 2-quinoxalin-2-yl-imidazo[1,2-a]pyridine-8-carboxamide; 2-naphth-2-yl-imidazo[1,2-a]pyridine-8-carboxamide; 2-naphth-1-yl-imidazo[1,2-a]pyridine-8-carboxamide; 2-(4-(2(N,N-dimethylamino)-eth-1-ylamino)-phenyl)-imidazo[1,2-a]pyridine-8-carboxamide; 2-(4-(2(N,N-diethylamino)-eth-1-ylamino)-phenyl)-imidazo[1,2-a]pyridine-8-carboxamide; 2-(4-(2-piperidin-1-yl-eth-1-ylamino)-phenyl)-imidazo[1,2-a]pyridine-8-carboxamide; 2-(4-(2-pyrrolidin-1-yl-eth-1-ylamino)-phenyl)-imidazo[1,2-a]pyridine-8-carboxamide; 2-(4-(3(N,N-dimethylamino)-prop-1-ylamino)-phenyl)-imidazo[1,2-a]pyridine-8-carboxamide; 2-(4-(3 (N,N-diethylamino)-prop-1-ylamino)-phenyl)-imidazo[1,2-a]pyridine-8-carboxamide; 2-(4-(3-piperidin-1-yl-prop-1-ylamino)-phenyl)-imidazo[1,2-a]pyridine-8-carboxamide; 2-(4-(3-pyrrolidin-1-yl-prop-1-ylamino)-phenyl)-imidazo-[1,2-a]pyridine-8-carboxamide; 2-cyclohexyl-imidazo[1,2-a]pyridine-8-carboxamide; 2-(cis-4-amino-cyclohex-1-yl)-imidazo[1,2-a]pyridine-8-carboxamide; 2-(4-methoxy-cyclohex-1-yl)-imidazo[1,2-a]pyridine-8-carboxamide; 2-(4-(2(N,N-dimethylamino)-eth-1-yl-methylamino)-phenyl)-imidazo[1,2-a]-pyridine-8-carboxamide; and 2-(4-(4-methyl-piperazin-1-yl)-phenyl)-imidazo[1,2-a]pyridine-8-carboxamide.

26. The method of claim 20, wherein the inhibitor of PARP activity comprises at least a compound selected from the group consisting of 3,4-dihydropyrrolo[4,3,2-de]isoquinolin-5-(1H)-one; 2-bromo-3,4-dihydropyrrolo[4,3,2-de]isoquinolin-5-(1H)-one; phenyl-3,4-dihydropyrrolo[4,3,2-de]isoquinolin-5-(1H)-one; 3,4,5,6-tetrahydro-1H-azepino[5,4,3-cd]indol-6-one; 2-bromo-3,4,5,6-tetrahydro-1H-azepino[5,4,3-cd]indol-6-one; 2-phenyl-3,4,5,6-tetrahydro-1H-azepino[5,4,3-cd]indol-6-one; 2-(4-Methoxyphenyl)-3,4,5,6-tetrahydro-1H-azepino[5,4,3-cd]indol-6-one; 2-(3-nitrophenyl)-3,4,5,6-tetrahydro-1H-azepino[5,4,3-cd]indol-6-one; 2-(3-hydroxymethylphenyl)-3,4,5,6-tetrahydro-1H-azepino[5,4,3-cd]indol-6-one; 2-(phenylethynyl)-3,4,5,6-tetrahydro-1H-azepino[5,4,3-cd]indol-6-one; 1-methyl-2-phenyl-3,4,5,6-tetrahydro-1H-azepino[5,4,3-cd]indol-6-one; 1-N-methyl-3,4,5,6-tetrahydro-1H-azepino[5,4,3-cd]indol-6-one; (rac)-3-phenyl-3,4,5,6-tetrahydro-1H-azepino[5,4,3-cd]indol-6-one; 2-(4-fluorophenyl)-3,4,5,6-tetrahydro-1H-azepino[5,4,3-cd]indol-6-one; 8-bromo-2-phenyl-3,4,5,6-tetrahydro-1H-azepino[5,4,3-cd]indol-6-one; 2-(4-(N,N-dimethylamino)methylphenyl)-3,4,5,6-tetrahydro-1H-azepino[5,4,3-c d]indol-6-one; 2-(3-(N,N-dimethylamino)methylphenyl)-3,4,5,6-tetrahydro-1H-azepino[5,4,3-c d]indol-6-one; 1,5-dihydro-[1,2]diazepino[4,5,6-cd]-indol-6-one; 1,5-dihydro-3-phenyl-[1,2]diazepino[4,5,6-cd]-indol-6-one; 1,5-dihydro-3-phenethyl-[1,2]diazepino[4,5,6-cd]-indol-6-one; 2-(3-trifluoromethyl-phenyl)-1,3,4,5-tetrahydro-azepino[5,4,3-cd]indol-6-one; 2-(4-trifluoromethyl-phenyl)-1,3,4,5-tetrahydro-azepino[5,4,3-cd]indol-6-one; 2-benzofuran-2-yl-1,3,4,5-tetrahydro-azepino[5,4,3-cd]indol-6-one; 2-(3,5-bis-trifluoromethyl-phenyl)-1,3,4,5-tetrahydro-azepino[5,4,3-cd]indo 1-6-one; 2-(4-bromophenyl)-1,3,4,5-tetrahydro-azepino[5,4,3-cd]indol-6-one; 2-(3-chloro-4-fluoro-phenyl)-1,3,4,5-tetrahydro-azepino[5,4,3-cd]indol-6-one; 2-(4-tert-butyl-phenyl)-1,3,4,5-tetrahydro-azepino[5,4,3-cd]indol-6-one; 2-phenyl-1,3,4,5-tetrahydro-azepino[5,4,3-cd]indole-6-thione; 2-phenethyl-1,3,4,5-tetrahydro-azepino[5,4,3-cd]indol-6-one; 2-(2-chlorophenyl)-1,3,4,5-tetrahydro-azepino[5,4,3-cd]indol-6-one; 2-naphthalen-1-yl-1,3,4,5-tetrahydro-azepino[5,4,3-cd]indol-6-one; 6-oxo-3,4,5,6-tetrahydro-1H-azepino[5,4,3-cd]indole-2-carboxylic acid methyl ester; 2-iodo-1,3,4,5-tetrahydro-azepino[5,4,3-cd]indol-6-one; 2-(4-(N-methylamino)methylphenyl)-3,4,5,6-tetrahydro-1H-azepino[5,4,3-cd]indol-6-one; 2-(3-(N-methylamino)methylphenyl)-3,4,5,6-tetrahydro-1H-azepino[5,4,3-cd]indol-6-one; 2-(3-(3-piperidin-1-ylmethylphenyl)-3,4,5,6-tetrahydro-1H-azepino[5,4,3-cd]indol-6-one; N-[3-(6-oxo-3,4,5,6-tetrahydro-1H-azepino[5,4,3-cd]indol-2-yl)-phenyl]-acetamide; 3-[2-(6-oxo-3,4,5,6-tetrahydro-1H-azepino[5,4,3-cd]indol-2-yl)-phenyl]-propionic acid methyl ester; 2-pyridin-3-yl-1,3,4,5-tetrahydro-azepino[5,4,3-cd]indol-6-one; 2-(2-methylsulfanyl-phenyl)-1,3,4,5-tetrahydro-azepino[5,4,3-cd]indol-6-one; 2-[4-(2-pyrrolidin-1-yl-ethyl)-phenyl]-1,3,4,5-tetrahydro-azepino[5,4,3-cd]indol-6-one; N-[4-fluoro-2-(6-oxo-3,4,5,6-tetrahydro-1H-azepino[5,4,3-cd]indol-2-yl)-phenyl]-acetamide; 6-oxo-3,4,5,6-tetrahydro-1H-azepino[5,4,3-cd]indole-2-carboxylic acid methylamide; 4-(6-oxo-3,4,5,6-tetrahydro-1H-azepino[5,4,3-cd]indol-2-yl)-benzoic acid; 6-oxo-3,4,5,6-tetrahydro-1H-azepino[5,4,3-cd]indole-2-carboxylic acid; 6-oxo-3,4,5,6-tetrahydro-1H-azepino[5,4,3-cd]indole-2-carboxylic acid (4-fluoro-phenyl)-amide; 2-(1H-pyrrol-2-yl)-1,3,4,5-tetrahydro-azepino[5,4,3-cd]indol-6-one; (RS)-(.+−.)-1-(4-chloro-phenyl)-9-(4-methoxy-phenyl)-8,9-dihydro-7H-2,7,9a-triaza-benzo[cd]azulen-6-one; (3-[1,3]dioxolan-2-yl-phenyl)-8,9-dihydro-7H-2,7,9a-triaza-benzo[cd]azulen-6-one; 1-(4-diethoxymethyl-phenyl)-8,9-dihydro-7H-2,7,9a-triaza-benzo[cd]azulen-6-one; 1-(3-pyrrolidin-1-ylmethyl-phenyl)-8,9-dihydro-7H-2,7,9a-triaza-benzo[cd]azulen-6-one; 1-[3-(3-trifluoromethyl-phenoxy)-phenyl]-8,9-dihydro-7H-2,7,9a-triaza-benzo[cd]azulen-6-one; 1-(3-[1,3]dioxolan-2-yl-phenyl)-4-fluoro-8,9-dihydro-7H-2,7,9a-triaza-benzo[cd]azulen-6-one; 1-(3-dimethylaminomethyl-phenyl)-4-fluoro-8,9-dihydro-7H-2,7,9a-triaza-benzo[cd]azulen-6-one; 1-(4-morpholin-4-ylmethyl-phenyl)-8,9-dihydro-7H-2,7,9a-triaza-benzo[cd]azulen-6-one; 1-(3-p-tolyl-benzo[c]isoxazol-5-yl)-8,9-dihydro-7H-2,7,9a-triaza-benzo[cd]azulen-6-one; 4-[5-(6-oxo-6,7,8,9-tetrahydro-2,7,9a-triaza-benzo[cd]azulen-1-yl)-pyridin-2-yloxy]-benzonitrile; 6-oxo-6,7,8,9-tetrahydro-2,7,9a-triaza-benzo[cd]azulen-1-carboxylic acid benzylamide; 1-(4-methyl-piperazine-1-carbonyl)-8,9-dihydro-7H-2,7,9a-triaza-benzo[cd]azulen-6-one; 1-[4-(2-pyrrolidin-1-yl-ethyl)-phenyl]-8,9-dihydro-7H-2,7,9a-triaza-benzo[c d]azulen-6-one; 1-[4-((2S,5S)-2,5-bis-methoxymethyl-pyrrolidin-1-ylmethyl)-phenyl]-8,9-dihydro-7H-2,7,9a-triaza-benzo[cd]azulen-6-one; and (R)-1-(4-dimethylaminomethyl-phenyl)-8-hydroxymethyl-8,9-dihydro-7H-2,7,9a-triaza-benzo[cd]azulen-6-one.

27. The method of claim 20, wherein the inhibitor of PARP activity comprises at least a compound selected from the group consisting of N-[3-(4-oxo-3,4-dihydro-phthalazin-1-ylamino)-propyl]-3-[3-(1H-pyrrol-2-yl)-[1,2,4]oxadiazol-5-yl]-propionamide; N-[3-(4-oxo-3,4-dihydro-phthalazin-1-ylamino)-propyl]-3-(3-thiophen-3-yl-[1,2,4]oxadiazol-5-yl)-propionamide; 3-[3-(2-methyl-thiophen-3-yl)-[1,2,4]oxadiazol-5-yl]-N-[3-(4-oxo-3,4-dihydro-phthalazin-1-ylamino)-propyl]-propionamide; 3-[3-(4-methanesulfonylamino-phenyl)-[1,2,4]oxadiazol-5-yl]-N-[3-(4-oxo-3,4-dihydro-phthalazin-1-ylamino)-propyl]-propionamide; 3-{3-[4-(2-morpholin-4-yl-ethoxy)-phenyl]-[1,2,4]oxadiazol-5-yl}-N-[3-(4-oxo-3,4-dihydro-phthalazin-1-ylamino)-propyl]-propionamide; 3-[3-(4-bromo-phenyl)-[1,2,4]oxadiazol-5-yl]-N-[3-(4-oxo-3,4-dihydro-phthalazin-1-ylamino)-propyl]-propionamide; 3-[3-(1,5-dimethyl-1H-pyrrol-2-yl)-[1,2,4]oxadiazol-5-yl]-N-[3-(4-oxo-3,4-dihydro-phthalazin-1-ylamino)-propyl]-propionamide; N-[2-hydroxy-3-(4-oxo-3,4-dihydro-phthalazin-1-ylamino)-propyl]-3-[3-(4-methylsulfanyl-phenyl)-[1,2,4]oxadiazol-5-yl]-propionamide; 3-[3-(4-fluorophenyl)-[1,2,4]oxadiazol-5-yl]-N-[2-hydroxy-3-(4-oxo-3,4-dihydro-phthalazin-1-ylamino)-propyl]-propionamide; 3-[3-(2,3-dihydro-benzofuran-5-yl)-[1,2,4]oxadiazol-5-yl]-N-[3-(4-oxo-3,4-dihydro-phthalazin-1-ylamino)-propyl]-propionamide; 1-[3-(5-oxo-5,6-dihydro-pyrido[2,3-d]pyridazin-8-ylamino)-propyl]-3-(2-oxo-tetrahydrofuran-3-yl)-thiourea; 3-[3-(6-dimethylamino-pyridin-3-yl)-[1,2,4]oxadiazol-5-yl]-N-[3-(4-oxo-3,4-dihydro-phthalazin-1-ylamino)-propyl]-propionamide; 3-[3-(4-acetylamino-phenyl)-[1,2,4]oxadiazol-5-yl]-N-[3-(4-oxo-3,4-dihydro-phthalazin-1-ylamino)-propyl]-propionamide; 5-oxo-5,6-dihydro-pyrido[2,3-d]pyridazin-8-ylamino)-cyclohexyl]-propionamide; [3-(5-oxo-5,6-dihydro-pyrido[2,3-d]pyridazin-8-ylamino)-propyl]-amide; 2-hydroxy-4-methylsulfanyl-N-[3-(5-oxo-5,6-dihydro-pyrido[2,3-d]pyridazin-8-ylamino)-propyl]-butyramide; 2-hydroxy-4-methylsulfanyl-N-[3-(8-oxo-7,8-dihydro-pyrazino[2,3-d]pyridazin-5-ylamino)-propyl]-butyramide; N-[3-(5-oxo-5,6-dihydro-pyrido[2,3-d]pyridazin-8-ylamino)-cyclohexyl]-propionamide; N-[2,2-dimethyl-3-(4-oxo-3,4-dihydro-phthalazin-1-ylamino)-propyl]-4-[3-(4-methoxy-phenyl)-[1,2,4]oxadiazol-5-yl]-butyramide; N-[3-(4-oxo-3,4-dihydro-phthalazin-1-ylamino)-propyl]-4-(3-pyridin-2-yl-[1,2,4]oxadiazol-5-yl)-butyramide; 4-[3-(3-nitro-phenyl)-[1,2,4]oxadiazol-5-yl]-N-[2-(4-oxo-3,4-dihydro-phthalazin-1-ylamino)-ethyl]-butyramide; 4-(3-{[3-(4-tert-butyl-phenyl)-[1,2,4]oxadiazol-5-ylmethyl]-amino}-propylamino)-2H-phthalazin-1-one; and 4-[3-(3,5-dichloro-phenyl)-[1,2,4]oxadiazol-5-yl]-N-[2-(4-oxo-3,4-dihydro-phthalazin-1-ylamino)-ethyl]-butyramide.

28. The method of claim 20, wherein the inhibitor of PARP activity comprises at least a compound selected from the group consisting of 2-(cis-4-amino-1-cyclohexyl)benzimidazole-4-carboxamide HCl; 2-(3-methoxycyclohexyl)benzimidazole-4-carboxamide; 2(4-methoxycyclohexyl)benzimidazole-4-carboxamide; 2-(4-(2-(N,N-diethylamino)ethoxy)cyclohexyl)benzimidazole-4-carboxamide 2HCl; trans-2-(4-aminocyclohexyl)benzimidazole-4-carboxamide; trans-2-(4-(aminomethyl)cyclohexyl)benzimidazole-4-carboxamide; 2-(4-methylcyclohexyl)benzimidazole-4-carboxamide; 2-(3-methylcyclohexyl)benzimidazole-4-carboxamide; 2-(2-methylcyclohexyl)benzimidazole-4-carboxamide; 2-(3-benzyloxyaminocyclohexyl)benzimidazole-4-carboxamide; 2-(3-aminocyclohexyl)benzimidazole-4-carboxamide HCl; 2-(cis-4-pyrrolidin-1-yl-1-cyclohexyl)benzimidazole-4-carboxamide; 2-(trans-4-pyrrolidin-1-yl-1-cyclohexyl)benzimidazole-4-carboxamide; 2-(cis-4-(piperazin-1-yl-1-cyclohexyl)benzimidazole-4-carboxamide; 2-(trans-4-(piperazin-1-yl-1-cylcyclohexyl)benzimidazole-4-carboxamide; 2-(4-(4-benzylpiperazin-1-yl-1-cyclohexyl)benzimidazole-4-carboxamide; 2-(3-(4-phenylpiperazin-1-yl-1-cyclohexyl)benzimidazole-4-carboxamide; 2-(3-(4-homopiperazin-1-yl-1-cyclohexyl)benzimidazole-4-carboxamide; 2-(4-(4-(N-methylhomopiperazin-1-yl)-1-cyclohexyl)benzimidazole-4-carboxamide; and 2-(3-(4-(4-phenyl-1,2,5,6-tetrahydropyridin-1-yl-1-cyclohexyl)benzimidazole-4-carboxamide.

29. The method of claim 20, wherein the composition further comprises a modulator of a pro-inflammatory gene expression.

30. The method of claim 29, wherein the modulator of a pro-inflammatory gene expression comprises an immunosuppressive medicament.

31. The method of claim 30, wherein the immunosuppressive medicament comprises a material selected from the group consisting of Cyclosporine, Azathioprine, Cyclophosphamide, Tacrolimus Hydrate, Mycophenolate Mofetil, Mycophenolic Acid, Pimecrolimus, Sirolimus, Pimecrolimus Hydrate, Sirolimus Hydrate, immunoglobulin antibodies, combinations thereof, and mixtures thereof.

32. The method of claim 30, wherein the immunosuppressive medicament comprises Cyclosporine A.

33. The method of claim 29, wherein the modulator of a pro-inflammatory gene expression comprises a dissociated glucocorticoid receptor agonist (“DIGRA”).

34. The method of claim 33, wherein the DIGRA comprises a compound having Formula XX or XXI 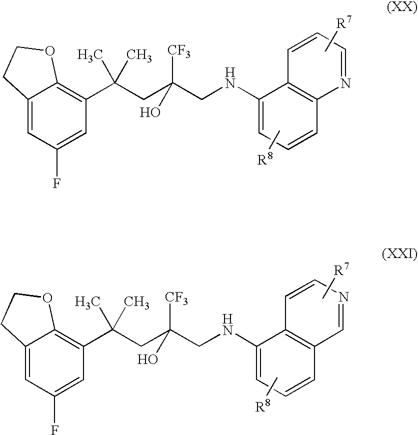
wherein R⁷ and R⁸ are independently selected from the group consisting of hydrogen, halogen, cyano, hydroxy, C₁-C₁₀ alkoxy groups, unsubstituted C₁-C₁₀ linear or branched alkyl groups, substituted C₁-C₁₀ linear or branched alkyl groups, unsubstituted C₃-C₁₀ cyclic alkyl groups, and substituted C₃-C₁₀ cyclic alkyl groups.

35. The method of claim 33, wherein the DIGRA comprises a compound having Formula XXII

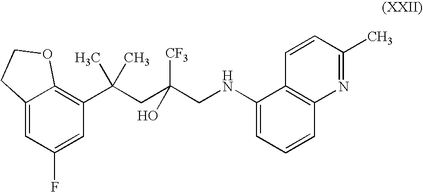

36. The method of claim 20, wherein said inhibitor of PARP activity is present in an amount in a range from about 0.001 to about 10 percent by weight of the total composition.

37. The method of claim 20, wherein said inhibitor of PARP activity is present in an amount in a range from about 0.01 to about 2 percent by weight of the total composition.

38. The method of claim 20, wherein the composition comprises a solution, dispersion, emulsion, suspension, or implant.

39. A method for preparing a composition that is suitable for the treatment, reduction, amelioration, alleviation, or prevention of a dry eye condition or a condition that requires the rewetting of the eye; the method comprising combining an inhibitor of PARP activity with a pharmaceutically acceptable carrier to form the composition.

40. The method of claim 39, further combining an additional material into the composition, wherein the additional material is selected from the group consisting of surfactants, buffers, diluents, adjuvants, expcipients, preservatives, co-solvents, oils, humectants, emollients, stabilizers, antioxidants, and combinations thereof.

41. The method of claim 39, further combining a modulator of pro-inflammatory gene expression into the composition.
